(19) United States
(12) Patent Application Publication
(10) Pub. No.: US 2025/0000650 A1
(43) Pub. Date: Jan. 2, 2025

(54) VALVE DELIVERY SYSTEM AND METHODS

(71) Applicant: Trisol Medical Ltd., Yokneam (IL)

(72) Inventors: Eli BEN-HAMOU, Bitan Aharon (IL); Nadav LEV, Hadera (IL); Mordehay VATURI, Ganei-Tikva (IL); Ron DAVIDSON, Hod-HaSharon (IL)

(73) Assignee: Trisol Medical Ltd., Yokneam (IL)

(21) Appl. No.: 18/704,973

(22) PCT Filed: Oct. 18, 2022
(86) PCT No.: PCT/IL2022/051095

§ 371 Date: Apr. 26, 2024

Related U.S. Application Data
(60) Provisional application No. 63/273,970, filed on Oct. 31, 2021.

(51) Int. Cl. A61F 2/24

(52) U.S. Cl. CPC A61F 2/2436

(57) ABSTRACT

The invention relates to a delivery system for a prosthetic heart valve, comprising an elongated body comprising a proximal end, a distal end and at least two bending portions, the two bending portions having different bending directions; an inner tube comprising a proximal end and a distal end, and positioned inside said outer tube; an operational distal end located at the distal end of the outer tube; the operational distal end comprising: an implant holder comprising at least two implant leg holders, one configured for holding one of said legs in a more axial proximally position than the other; an atraumatic distal tip connected to a distal end of the implant holder; and an implant enclosure for externally enclosing the implant, proximally connected to the distal end of the outer tube, and extending until a proximal end of the distal tip.

### RELATED APPLICATION/S

This application claims the benefit of priority of U.S. Provisional Patent Application No. 63/273,970 filed on 31 Oct. 2021, the contents of which are incorporated herein by reference in their entirety.

### FIELD AND BACKGROUND

The present invention, in some embodiments thereof, relates to a valve delivery system and related methods and, more particularly, but not exclusively, to a prosthetic replacement for a tricuspid valve delivery system and related methods.

Tricuspid Regurgitation (TR) occurs when the tricuspid valve fails to close properly, causing blood to flow backward from the Right Ventricle to the right atrium. Many cases of Tricuspid Regurgitation are accompanied by the Dysfunction of the Right Ventricle.

Treating patients with TR often require valve replacement in order to reduce or eliminate the regurgitation. In recent years, a trans-vascular technique has been developed for introducing and implanting a prosthetic heart valve using a flexible catheter in a manner that is less invasive than open heart surgery.

In the trans-vascular technique, the prosthetic is delivered to the target site (i.e.: tricuspid valve) through a catheter while the device is crimped to a low diameter shaft, and when it is located in the correct position it is expanded/deployed to the functional size. During the positioning of the device, the heart is still functioning, meaning, the heart is pumping, the blood is flowing and everything is moving while the physician tries to position the device in the optimal position. In addition to this, the anatomical configuration of the heart does not allow a direct and easy approach of the device and the implant to the required location. Current devices do not allow an easy and fine tuning of the positioning of the device during implantation.

Additional background art includes U.S. Patent No. U.S. Ser. No. 10/137,019B2 disclosing a medical instrument including “a first rod; a second rod which extends along the first rod; a longitudinal center line defined by the rods; and a manipulator. The manipulator includes a plurality of fingers, each with a first finger end and a second finger end. The second finger ends are free ends. The first finger ends are supported on the second rod in such a way that the fingers are operable, by moving the second rod in relation to the first rod, in order to be displaced from a first position to a second position. The distance from the free ends of the fingers to the longitudinal center line in the first position is different compared with the second position. A stent, a ring prosthesis and a prosthesis device are also described.”

International Patent Application Publication No. WO2019086958A1 disclosing “device assemblies for endovascular prosthesis implantation in a human patient comprising a collapsed delivery configuration and an expanded deployed configuration comprising: a stent frame comprising a plurality of expandable stent cells arranged in more than one layer and stacked in a longitudinal direction; a valve attached to the stent frame; and an expandable sealing skirt coupled to the stent frame, the expandable sealing skirt comprising a protruding region configured to expand radially past the stent frame when the device assembly is in the expanded deployed configuration and a non-protruding region.”

U.S. Patent No. U.S. Pat. No. 9,763,778B2 disclosing “an anchoring device for use within a cardiovascular structure includes an expandable ring having a central axis extending in a longitudinal direction. At least one leg extends from the ring in the longitudinal direction. The leg includes a first end connected to the ring and a free end, with at least one anchor connected to the leg. The anchoring device may be used to anchor a transcatheter heart valve within the cardiovascular structure.”

U.S. Patent No. U.S. Pat. No. 7,402,151B2 disclosing “methods for easy, atraumatic access to areas of the vasculature that are otherwise difficult to access, using steerable guide catheters constructed with components that are selected to provide optimal navigability, torque transfer, and push ability for a variety of typical percutaneous access routes. The catheter wall thickness in the deflecting segment of the guide catheter is about 1 French (⅓ mm) or less, and includes a slotted deflection tube, and this construction allows a very tight turning radius which in turn enables guide catheter access to regions of the vasculature that are otherwise inaccessible.”

International Patent Application Publication No. WO2019038337A1 disclosing a double steerable sheath, a delivery system comprising such a sheath and a method for deployment of a medical device, e.g. a replacement heart valve prosthesis, in a patient's heart.

U.S. Patent No. U.S. Pat. No. 8,096,985B2 disclosing “devices and methods for guide catheters having one or more regions of increased flexibility. A flexibility region comprises one tubular segment of the guide catheter with a non-linear longitudinal seam between two non-concentric layers of material having different durometers. A non-linear seam, such as a zig-zag or sinusoidal configuration, permits controlled compression of lower durometer material between portions of higher durometer material.”

International Patent Application Publication No. WO2018174712A1 disclosing “a guide wire-catheter assembly, comprising: a catheter tube having a longitudinal channel, and a guide wire configured to be movable in the longitudinal channel characterized in that, the catheter tube comprises a bendable part near its distal end, wherein the bendable part comprises, in the circumferential direction of the catheter tube, a varying flexibility, such that exerting a longitudinal compression force in a proximal direction at a compression location distally of the distal end part results in bending of the bendable part, and in that the guide wire comprises an expandable part, wherein the expandable part is movable between a non-expanded position, in which a cross section of the expandable part is smaller than a smallest cross section of the longitudinal channel of the catheter tube and an expanded position, in which a cross section of the expandable part is larger than the smallest cross section of the longitudinal channel of the catheter tube, wherein, when the expandable part is in the non-expanded position, the guide wire can be moved completely in and out of the longitudinal channel of the catheter tube, and wherein, when the expandable part is in the non-expanded position, the expandable part is configured to exert the longitudinal compression force on the compression location in order to bend the bendable part of the catheter tube.”

U.S. Patent No. U.S. Pat. No. 6,869,414B2 discloses “a catheter employs a pre-formed distal end and a proximal deflection mechanism for steering the catheter. A shaped member extends from a shaped region of the catheter sheath to at least a portion of an anchor region. A steering ribbon extends from a proximal region of the sheath and passes within at least a portion of the anchor region. A distal end of the steering ribbon is joined with a proximal portion of the shaped member. At least one steering tendon is disposed within the sheath and has a first end attached at the anchor region and a second end located at the proximal region of the sheath. Movement of the steering tendon in a proximal direction causes the deflection region to deflect relative to the longitudinal axis of the catheter while the shape of shaped region of the sheath is substantially maintained.”

European Patent No. EP0980693A1 disclosing “a bi-directional electrode catheter comprising an elongated tubular catheter body, a catheter tip section at the distal end of the catheter body and a control handle at the proximal end of the catheter. The tip section comprises two pair of generally diametrically opposed off-axis lumens. Two pair of puller wires extend from the handle, through the catheter body, and into the off-axis pair of lumens of the tip section, where they are anchored in the tip section at different locations along the length of the tip section. Compression coils extend through the catheter body in surrounding relation to the puller wires. At their proximal ends, the puller wires are attached to movable pistons in the control handle. Each piston is controlled by an operator using a slidable button fixedly attached to each piston. Movement of selected buttons results in deflection of the tip section into a generally planar “U”- or “S”-shaped curve.

### SUMMARY

Following is a non-exclusive list including some examples of embodiments of the invention. The invention also includes embodiments which include fewer than all the features in an example and embodiments using features from multiple examples, also if not expressly listed below.

Example 1. A delivery system for a prosthetic heart valve, comprising:
a. an elongated body comprising a proximal end, a distal end and at least two bending portions, said two bending portions having different bending directions;
b. an inner tube comprising a proximal end and a distal end, and positioned inside said outer tube;
c. an operational distal end located at said distal end of said outer tube; said operational distal end comprising:
i. an implant holder reversibly attached to said distal end of said inner tube; said implant holder comprising at least two implant leg holders, one configured for holding one of said legs in a more axial proximally position than the other;
ii. an atraumatic distal tip connected to a distal end of said implant holder; and
iii. an implant enclosure for externally enclosing said implant, proximally connected to said distal end of said outer tube, and extending until a proximal end of said distal tip.

Example 2. The delivery system according to example 1, wherein said elongated body comprises external flexible tube.

Example 3. The delivery system according to example 1 or example 2, wherein said bending portions are configured to bent to a bending angle of from about 0 degrees to about 180 degrees.

Example 4. The delivery system according to any one of examples 1-3, wherein said external flexible tube is made of one or more of braided Pebax, PEEK and stainless steel.

Example 5. The delivery system according to any one of examples 1-4, wherein said elongated body is long enough to reach a heart of a patient from the outside of the body of said patient using a femoral entry or a jugular entry or another entry.

Example 6. The delivery system according to any one of examples 1-5, wherein said elongated body comprises a port to be located at an incision point in said body of said patient.

Example 7. The delivery system according to any one of examples 1-6, wherein said elongated body is configured to move along said port.

Example 8. The delivery system according to any one of examples 1-7, wherein said port comprises and inlet-outlet tube for inserting and removing liquids from a blood vessel.

Example 9. The delivery system according to any one of examples 1-8, wherein said inner tube is made of one or more of braided Pebax, PEEK and stainless steel.

Example 10. The delivery system according to any one of examples 1-9, wherein said inner tube is one long tube.

Example 11. The delivery system according to any one of examples 1-10, wherein said one long tube comprises a plurality of zones having different levels of stiffness.

Example 12. The delivery system according to any one of examples 1-11, wherein said inner tube comprises two or more parts.

Example 13. The delivery system according to any one of examples 1-12, wherein said inner tube comprises two parts, a first part extending from a handle of said delivery system to a certain location of said elongated body and a second part extending from said certain location of said elongated body to said operational distal end.

Example 14. The delivery system according to any one of examples 1-13, wherein said second part is covered with an envelope.

Example 15. The delivery system according to any one of examples 1-14, wherein said first part is stiffer than said second part.

Example 16. The delivery system according to any one of examples 1-15, wherein said first part is from about 0.1% to about 50% stiffer than said second part.

Example 17. The delivery system according to any one of examples 1-16, wherein said second part is more flexible than said first part.

Example 18. The delivery system according to any one of examples 1-17, wherein said second part is from about 0.1% to about 50% more flexible than said first part.

Example 19. The delivery system according to any one of examples 1-18, wherein said implant holder is made of one or more of PEEK and stainless steel.

Example 20. The delivery system according to any one of examples 1-19, wherein said implant enclosure is transparent.

Example 21. The delivery system according to any one of examples 1-20, wherein said implant enclosure is sized and shaped to accommodate said prosthetic heart valve in a crimped configuration.

Example 22. The delivery system according to any one of examples 1-21, further comprising a handle at said proximal end of said elongated body for actuating said delivery system.

Example 23. The delivery system according to any one of examples 1-22, wherein said actuating comprises actuating said at least two bending portions.

Example 24. The delivery system according to any one of examples 1-23, wherein said actuating comprises moving axially said elongated body.

Example 25. The delivery system according to any one of examples 1-24, wherein said actuating comprises moving axially said inner tube.

Example 26. The delivery system according to any one of examples 1-25, wherein said actuating is performed manually by actuating one or more knobs.

Example 27. The delivery system according to any one of examples 1-26, wherein said actuating is performed electronically by activating one or more motors.

Example 28. The delivery system according to any one of examples 1-27, wherein said handle comprises one or more knobs for performing said actuating.

Example 29. The delivery system according to any one of examples 1-28, wherein said handle comprises a quick release knob in communication with said elongated body and configured to allow rotation of said elongated body along its longitudinal axis by rotating of said quick release knob.

Example 30. The delivery system according to any one of examples 1-29, wherein said handle comprises an inlet/outlet tube configured to allow the insertion/extraction of matter from said delivery system.

Example 31. A method of delivering a prosthetic heart valve with a delivery system in a valve area located between an atrial space and a ventricular space,
said delivery system comprising: an elongated body having a distal end comprising an operational distal end including said prosthetic heart valve in a crimped configuration,
a virtual chord being defined between two locations adjacent to said operational distal end; said virtual chord having a length;
the method comprising:
a. inserting said distal end into a heart of a subject;
b. positioning said operational distal end in said ventricular space passing through said atrial space and said valve area;
c. retracting said operational distal end into said valve area by deforming at least one portion of said shaft adjacent to said operational distal end; said deforming shortens said length of said virtual chord; wherein said deforming aligns said operational distal end ±20 degrees perpendicular to said opening of said heart valve.

Example 32. The method according to example 31, wherein said at least one portion of said shaft is located in said atrium space while said deforming.

Example 33. The method according to example 31 or example 32, wherein said positioning said operational distal end in said ventricular space comprises completely positioning said operational distal end in said ventricular space.

Example 34. The method according to any one of example 31-33, wherein said method further comprises actuating said operational distal end for releasing said prosthetic heart valve in said valve area.

Example 35. The method according to any one of examples 31-34, wherein said actuating comprises retracting proximally said elongated body.

Example 36. The method according to any one of examples 31-35, wherein said actuating comprises pushing distally a prosthetic heart valve holder located within said operational distal end.

Example 37. The method according to any one of examples 31-36, wherein said releasing comprises releasing a plurality of anchors of said prosthetic heart valve into said valve space at said ventricular space, while holding the rest of said prosthetic heart valve in said operational distal end.

Example 38. The method according to any one of examples 31-37, wherein said releasing further comprises, after said releasing of said plurality of anchors, releasing one or more prosthetic heart valve leg holders, while holding at least one of said prosthetic heart valve leg holders of said prosthetic heart valve in said operational distal end.

Example 39. The method according to any one of examples 31-38, wherein said releasing further comprises manipulating said elongated body for further positioning said prosthetic heart valve in said valve area.

Example 40. The method according to any one of examples 31-39, wherein said releasing further comprises releasing said at least one of said prosthetic heart valve leg holders of said prosthetic heart valve, thereby completely releasing said prosthetic heart valve from said operational distal end and in said valve area.

Example 41. The method according to any one of examples 31-40, wherein said deforming comprises bending one or more location in said elongated body.

Example 42. The method according to any one of examples 31-41, wherein said deforming comprises bending two distinct locations in said elongated body.

Example 43. The method according to any one of examples 31-42, wherein said bending two distinct locations in said elongated body comprises bending two distinct location having opposite bending direction from each other.

Example 44. A method of delivering a prosthetic heart valve with a delivery system, said delivery system comprising an operational distal end including said heart valve in a crimped configuration, the method comprising:
a. inserting said delivery system into a heart of a subject;
b. bending at least a first part of said delivery system so as to position a part of said operational distal end of said delivery system within a ventricle of said heart;
c. bending at least a second part of said delivery system so as to position said part of said operational distal end of said delivery system within said valve between said ventricle and an atrium;
d. releasing said prosthetic heart valve within said valve.

Example 45. The method according to example 44, wherein said releasing said prosthetic heart valve within said valve comprises one or more of:
a. partially releasing said prosthetic heart valve;
b. correcting a positioning of said prosthetic heart valve;
c. completely release heart valve.

Example 46. The method according to example 44 or example 45, wherein said position said part of said operational distal end of said delivery system within said valve comprises positioning said prosthetic heart valve substantially perpendicular to a valve opening.

Example 47. The method according to any one of examples 44-46, wherein said first part of said delivery system and said second part of said delivery system are parts adjacent to said operational distal end.

Example 48. The method according to any one of examples 44-47, wherein said bending of said first part of said delivery system and said bending of said second part of said delivery system comprise bending said parts of said delivery system within said atrium of said heart.

Example 49. The method according to any one of examples 44-48, wherein said bending of said first part of said delivery system and said bending of said second part of said delivery system comprise bending said parts of said delivery system to opposite bending directions from each other.

Example 50. The method according to any one of examples 44-49, wherein said method further comprises extracting said delivery system after said releasing of said prosthetic heart valve.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1a is an exemplary prosthetic replacement for a tricuspid valve delivery system, according to some embodiments of the invention;

FIGS. 1b-1e are two exemplary prosthetic valve implants, according to some embodiments of the invention;

FIG. 2 is an illustration of an exemplary handle of the delivery catheter according to some embodiments of the invention;

FIG. 3 is an illustration of an exemplary elongated body, according to some embodiments of the invention;

FIGS. 4a-4c are illustrations of an exemplary implant housing/operational distal end, according to some embodiments of the invention;

FIGS. 5a-5b are illustrations of an exemplary implant holder, according to some embodiments of the invention;

FIGS. 6a-6k are illustrations of exemplary embodiments of the implant leg holder, according to some embodiments of the invention;

FIGS. 7a-7b are illustrations of an exemplary implant capsule/enclosure, according to some embodiments of the invention;

FIGS. 8a-8c are illustrations of different angles of an exemplary distal tip, according to some embodiments of the invention;

FIGS. 9a-9c are illustrations of exemplary steering movements of the distal end of the elongated body, according to some embodiments of the invention;

FIG. 9d is an illustration of an exemplary bending movement in zone 1204, according to some embodiments of the invention;

FIG. 9e is an illustration of an exemplary bending movement in both zone 1202 and zone 1204, according to some embodiments of the invention;

FIGS. 9f-9g are illustrations of an exemplary combination of movements of the distal end of the device, according to some embodiments of the invention;

FIGS. 9h-9i are schematic representations of definition of movements of the delivery device, according to some embodiments of the invention;

FIGS. 10a-10b are illustrations of an exemplary quick release knob, according to some embodiments of the present invention;

FIGS. 11a-11o are illustrations of an exemplary preparation procedure of the system according to some embodiments of the invention;

FIG. 12 is a schematic representation of the path that the delivery device performs during the implantation procedure, according to some embodiments of the invention;

FIGS. 13a-13g are illustrations of an exemplary delivery method of an implant having legs with the same length, according to some embodiments of the invention; and

FIGS. 14a-14d are illustrations of an exemplary delivery method of an implant having legs with different lengths, according to some embodiments of the invention.

### DESCRIPTION OF SPECIFIC EMBODIMENTS OF THE INVENTION

The present invention, in some embodiments thereof, relates to a valve delivery system and related methods and, more particularly, but not exclusively, to a prosthetic replacement for a tricuspid valve delivery system and related methods.

### Overview

An aspect of some embodiments of the invention relates to manipulating heart implants, which are configured to be placed inside the heart, during their deployment so as to facilitate the positioning of the implant inside the heart. In some embodiments, the heart implants are brought inside the heart in a compressed/crimped configuration in a delivery device. In some embodiments, the heart implants are first partially deployed to a first positioning state while keeping at least one part of the heart implant in communication with the delivery device. In some embodiments, keeping at least one part of the implant in communication with the delivery device allows the user to perform adjustments to the first positioning to provide a second positioning, which is a positioning as required for the implant and/or as desired by the user. In some embodiments, once the heart implant is positioned, the user completely deploys the heart implant by releasing the at least one part of the heart implant from the delivery device. In some embodiments, the partially deployment of the heart device is a controlled deployment, meaning that the deployment is controlled by the user and/or the delivery device is configured to release the heart implant so the expansion of the heart valve is performed to a predetermined direction and/or manner. In some embodiments, the delivery device is restricted a priori of levels of freedom so as to ease for the user the deployment of the heart implant. In some embodiments, the delivery device is configured to bend in two or more places, each in a predetermined direction, so as to provide the delivery device with a distal end having specific possibilities of movement. In some embodiments, the delivery system comprises a distal end comprising a plurality of parts configured for the transport and releasing of a heart implant. In some embodiments, the distal end comprises a housing/operational distal end for the heart implant configured to maintain the heart implant in a compressed/crimped configuration until the moment of deployment/partial deployment.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### General View of the Exemplary Delivery System

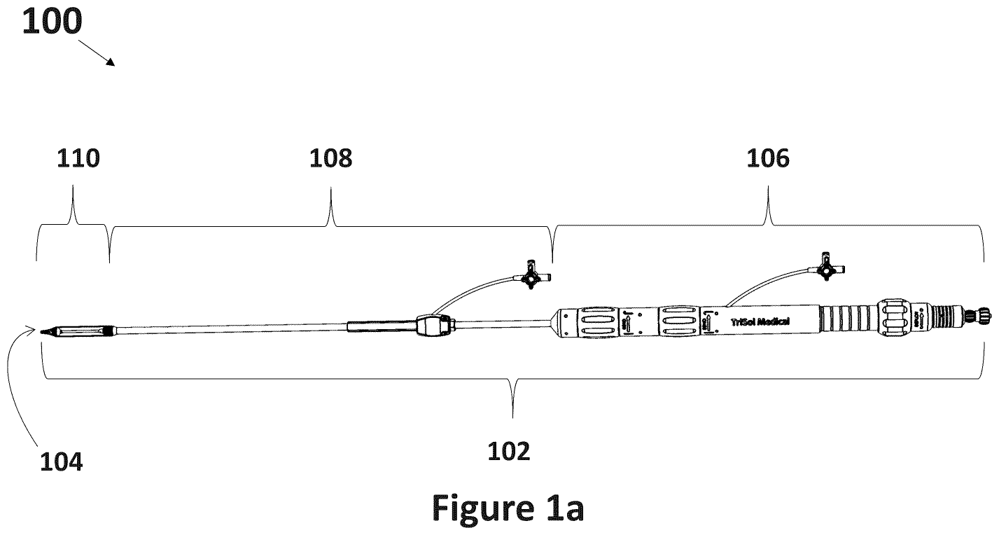

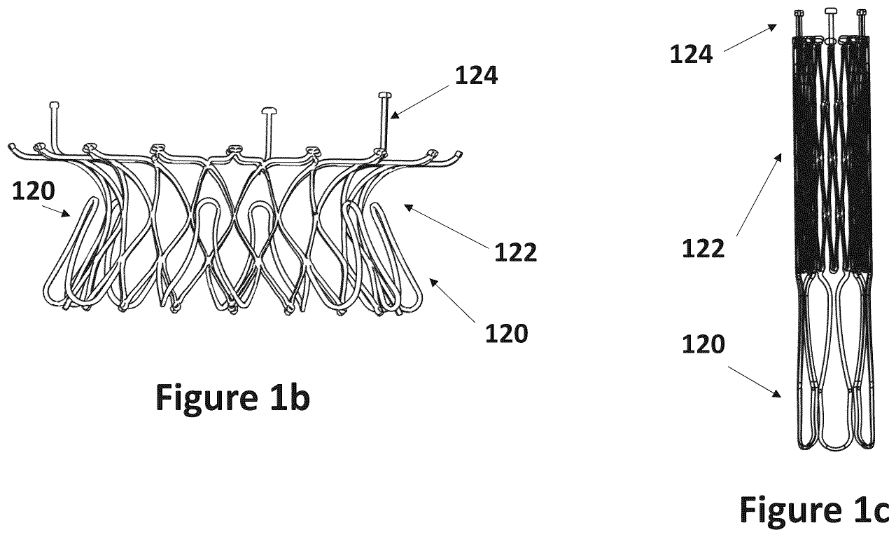

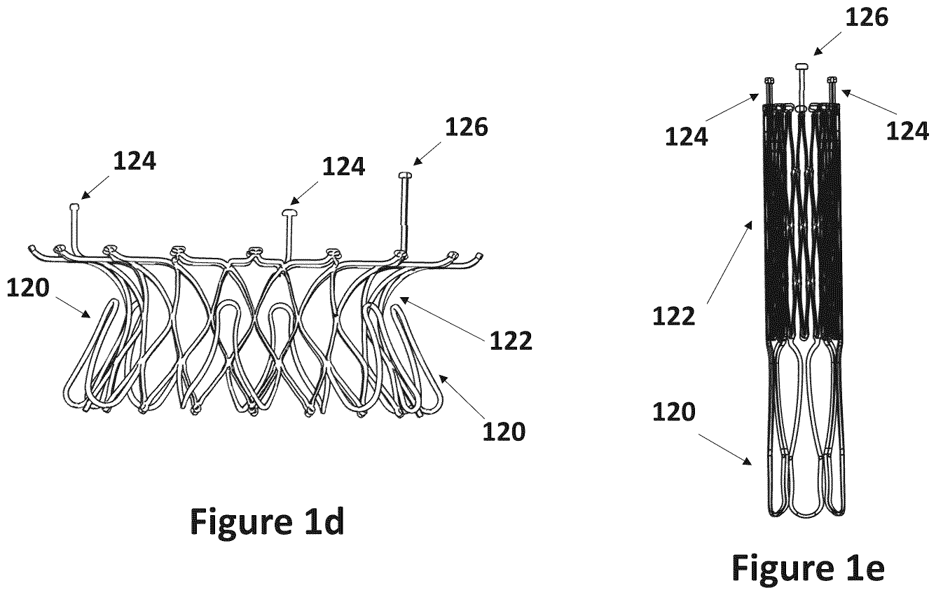

Referring now to the drawings, FIG. 1a illustrates an exemplary prosthetic replacement for a tricuspid valve delivery system, according to some embodiments of the invention. In some embodiments, the delivery system 100 comprises a catheter 102 on which, at its distal end, a prosthetic valve 104 is mounted. In some embodiments, the catheter 102 comprises a handle 106, an elongated body 108 extending from the distal end of the handle 106, and an operational distal end (also referred as implant housing) 110 located distally of the elongated body 108.

### Exemplary Implant

Referring now to FIGS. 1b-e, illustrating two exemplary implants 112, according to some embodiments of the invention. In order to facilitate the explanations that will follow, an exemplary implant 112 is shown, in an open configuration, as shown in FIGS. 1b and 1d, and in a compressed/crimped configuration, as shown in FIGS. 1c and 1e. FIGS. 1b-e show an incomplete implant where only the “skeleton” of the implant is shown. The scope of these figures is to identify the relevant parts involved in the deployment of the implant and that are in communication with the delivery system, which will help in the understanding of the invention. Roughly, the implant can be divided in 3 main parts: the anchors 120, the middle body 122 and the legs 124. As can be seen, in some embodiments, in the open configuration, the anchors 120 are folded towards the middle body 122, while in the crimped configuration the anchors 120 are extended down. It can also be seen that, in some embodiments, in the open configuration the middle body 122 is wide open, while in the crimped configuration the middle body 122 is thin and narrow. In some embodiments, the difference between the implant shown in FIGS. 1b-c and the implant shown in FIGS. 1d-e is that the implant shown in FIGS. 1d-e comprises 2 short legs 124 and one longer leg 126 (longer than the other two legs 124). In some embodiments, the short legs comprise a length of from about 3 mm to about 5 mm; optionally, from about 2.5 mm to about 5.5 mm; optionally, from about 1 mm to about 6 mm. In some embodiments, the long leg comprises a length that is longer than the short legs from about 1 mm to about 3 mm; optionally, from about 0.5 mm to about 3.5 mm; optionally, from about 0.3 mm to about 6 mm. The implant is made of self-expanding and/or shape memory material, for example nitinol, which allow crimping the implant for the delivery and once arrived at the location, while releasing the implant, the implant returns to the open configuration without any further help.

### Exemplary Handle 106

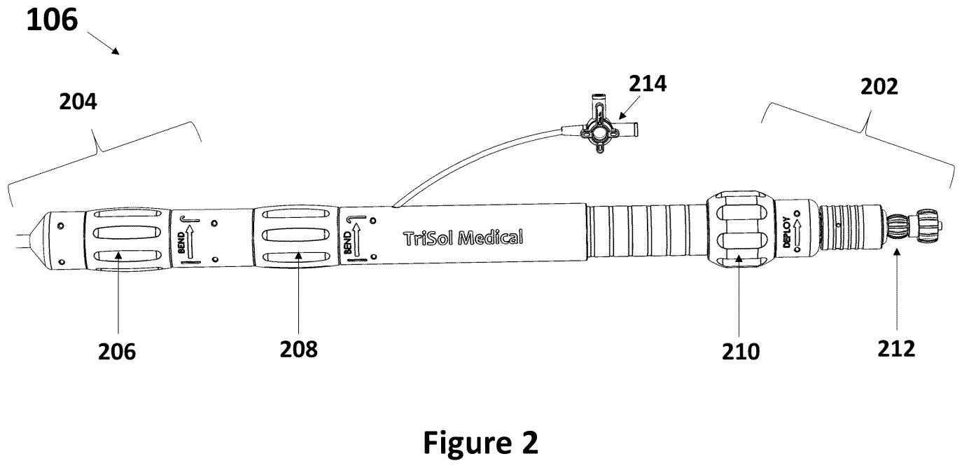

Referring now to FIG. 2, illustrating an exemplary handle 106 of the delivery catheter according to some embodiments of the invention. FIG. 2 illustrates a side view of an exemplary handle 106. In some embodiments, the handle is located at a proximal end of the delivery system 100, and it is operated from outside the body of the patient. In some embodiments, the handle comprises a proximal end 202 and a distal end 204. In some embodiments, the handle comprises at least one actuation mechanism configured to actuate one or more of a movement of the elongated body 108 and/or a movement of the implant housing/operational distal end 110 and/or a movement of an outer/external tube 302 of the elongated body and/or a movement of an internal/inner tube. In some embodiments, the actuation of the one or more of a movement of the elongated body 108 and/or a movement of the implant housing/operational distal end 110 and/or a movement of an outer/external tube 302 of the elongated body and/or a movement of an internal/inner tube is manually actuated; optionally the handle comprises one or more actuators, for example an electrical actuator and or a pneumatic actuator. For example, the exemplary handle illustrated in FIG. 2 show an exemplary handle where movements of the elongated body 108 and/or movements of the implant housing/operational distal end 110 and/or a movement of an outer/external tube 302 of the elongated body and/or a movement of an internal/inner tube are manually actuated. It should be understood that this example is provided to allow a person having skills in the art to understand the invention and should not be limiting in any way. In this example, handle 106 comprises 3 control knobs 206/208/210, for example 3 rotating knobs, and one quick release knob 212 (see below for further details about quick release knob 212). In this example, rotating knobs 206 and 208 are used to control two different movements of the elongated body 108, while rotating knob 210 is used to control the longitudinal movements of the elongated body 108. In some embodiments, knobs 206/208/210 can alternatively be controlled and rotated by an electric operated motor. In some embodiments, in addition, the internal tube can alternatively be pushed and pulled using an electric motorized lead screw, while the main handle remains fixed. In some embodiments, the handle comprises an inlet/outlet tube 214 configured to allow the insertion of liquids, for example saline, and the extraction of liquids and possible bubbles, for example blood or oxygen or air, from the delivery system 100 and more specifically from the implant housing/operational distal end 110. It should be understood that the aforementioned mechanisms for controlling the delivery device and for releasing the implant, are exemplary mechanisms disclosed to allow a person having skills in the art to understand the invention. Other mechanisms could be used, and those are also included in scope of the invention.

### Exemplary Elongated Body 108

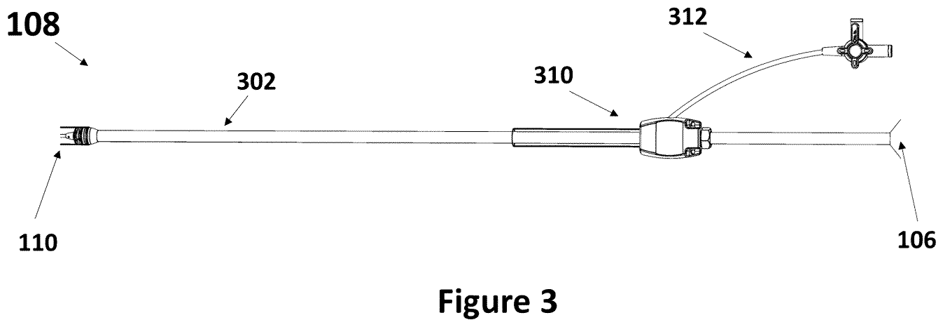

Referring now to FIG. 3, illustrating an exemplary elongated body 108, according to some embodiments of the invention. FIG. 3 illustrates a side view of the exemplary elongated body 108. In some embodiments, the catheter 102 comprises an elongated body 108 extending from the distal end of the handle 106 until the proximal end of the implant housing/operational distal end 110. In some embodiments, the elongated body 108 begins from the proximal end of the handle 106, in communication with a quick release knob 212 (see above and below), extends inside the handle 106 and exits the handle 106 at the distal end of the handle 106. In some embodiments, the elongated body 108 comprises an external flexible tube 302, optionally made of one or more of Braided Pebax/PEEK/Stainless steel. In some embodiments, the elongated body 108 comprises one long internal flexible tube (not shown), optionally made of one or more of Braided Pebax/PEEK/Stainless steel. In some embodiments, optionally, the internal tube comprises two or more parts, a first part extending from the handle 106 until a certain distance of the elongated body 108, and a second part extending until the implant housing/operational distal end 110. In some embodiments, the second part is covered with an envelope, optionally made of one or more of Pebax/PVC. In some embodiments, the first part is stiffer than the second part. In some embodiments, the first part is from about 1% to about 10% stiffer than the second part. Optionally from about 0.5% to about 20% stiffer. Optionally from about 0.1% to about 50% stiffer. In some embodiments, the second part is more flexible than the first part. In some embodiments, the second part is from about 1% to about 10% more flexible than the first part. Optionally from about 0.5% to about 20% more flexible. Optionally from about 0.1% to about 50% more flexible. In some embodiments, a potential advantage of having a two internal tubes having different levels of stiffness is providing a catheter 102 that comprises more flexible zones where necessary and more stiff zones where flexibility if not a requirement. In some embodiments, when a single internal tube is used, the internal tube comprises zones with different stiffness between each other, so as to provide the delivery device with the necessary strength where and when necessary and with the necessary flexibility when and where necessary. For example, an extruded Pebax tube with changing stiffness on the longitudinal sections. In some embodiments, rotating knob 210 (or any other actuation mechanism—see above) actuates the longitudinal movement of the external tube 202 alone, without moving the internal tube 204. In some embodiments, rotating knob 210 or any other actuation mechanism—see above) actuates the longitudinal movement of the internal tube alone, without moving the external tube 302. In some embodiments, rotating knob 210 or any other actuation mechanism—see above) actuates the longitudinal movement of the internal tube to one direction, while actuating the longitudinal movement of the external tube 302 to an opposite direction of that of the internal tube. For example, the internal tube moves distally while the external tube 302 moves proximally, and vice versa.

In some embodiments, the length of the elongated body is long enough so as to reach the heart from outside the body via either a femoral entry or a jugular entry or another entry.

In some embodiments, mounted on the elongated body 108 there is a port 310 configured to be located at the incision point in the body on the patient (i.e. the location where the physician cuts the patient), and stay put. In some embodiments, this enables the movement of the catheter 102 inside the port 310 without further damaging the blood vessels. In some embodiments, connected to the port 310 there is an inlet-outlet tube 312 configured to insert or remove liquids from the blood vessel.

### Exemplary Implant Housing/Operational Distal End 110

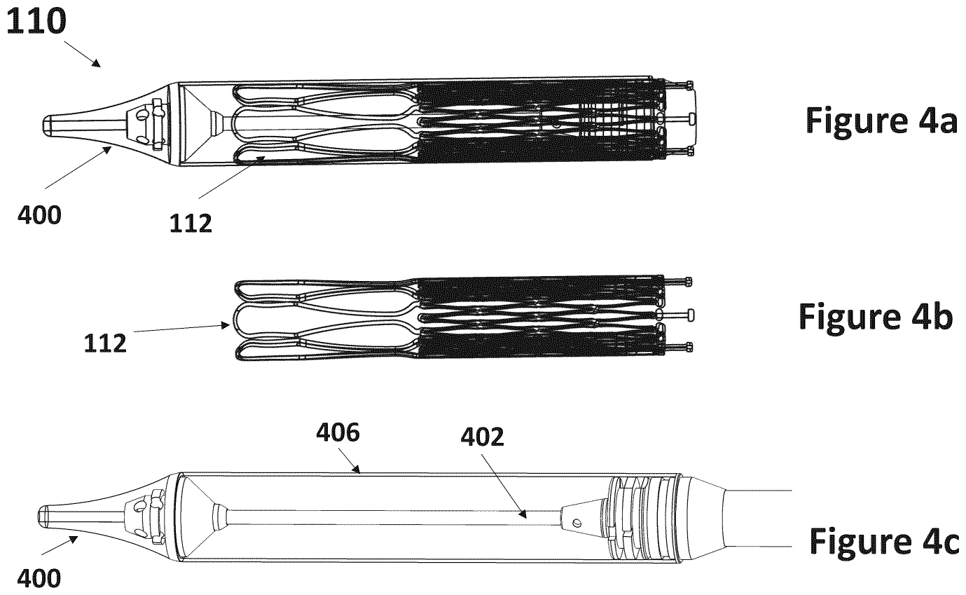

Referring now to FIGS. 4a-c, illustrating an exemplary implant housing/operational distal end 110, according to some embodiments of the invention. FIG. 4a, illustrates a side view of an exemplary implant housing/operational distal end 110 comprising a schematic implant 112 in a crimped configuration. FIG. 4b, illustrates the schematic implant 112 in a crimped configuration, outside the exemplary implant housing/operational distal end 110. It should be understood that the schematic implant 112 is shown here to facilitate the explanations of the delivery system 100 and should not limit the invention in any way. FIG. 4c, illustrates a side view of the exemplary implant housing/operational distal end 110 without the schematic implant 112, to allow the easy view of the parts.

In some embodiments, the implant housing/operational distal end 110 comprises the following parts: a distal tip 400, an implant holder 402, a connector (not shown) between the implant holder 402 and the internal tube of the elongated body 108 and an implant capsule/enclosure 406 mounted on the distal end of the external tube 302. In some embodiments, the different parts are connected to each other through screw/thread mechanisms. In some embodiments, a potential advantage of having screwable/threaded parts is that, on one side, provides a device that is easily mounted and/or armed by a user, while, on the other side, it provides a more reliable device during its use since the parts are tightly connected to each other.

### Exemplary Implant Holder 402

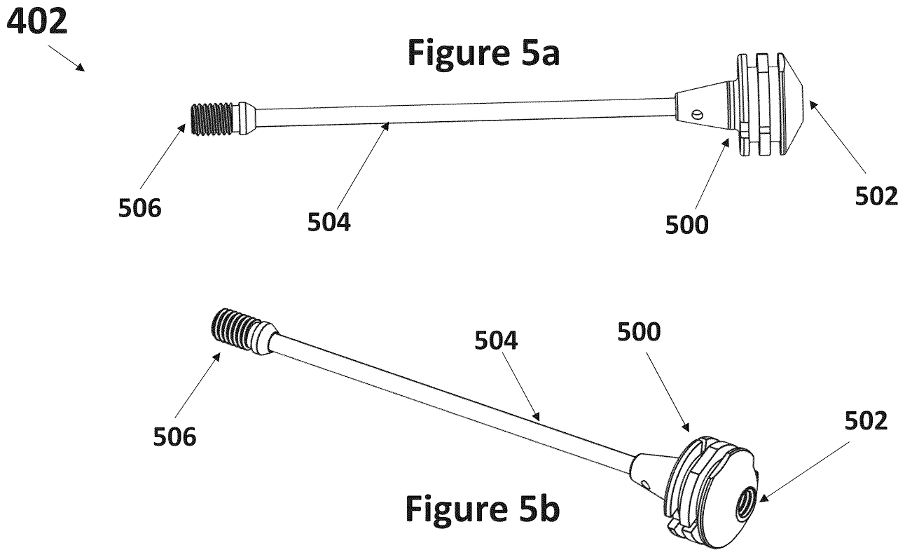

Referring now to FIGS. 5a-b, illustrating an exemplary implant holder, according to some embodiments of the invention. In some embodiments, the implant holder 402 comprises an implant leg holder 500 at the proximal end comprising an orifice 502 having internal threaded walls, a connector tube 504 comprising a threaded adaptor 506 located at the distal end. In some embodiments, orifice 502 is configured to be connected with the connector and therefore to the elongated body 108. In some embodiments, threaded adaptor 506 is configured to be connected with the distal tip 400. In some embodiments, the materials of the implant holder are one or more of PEEK/Stainless.

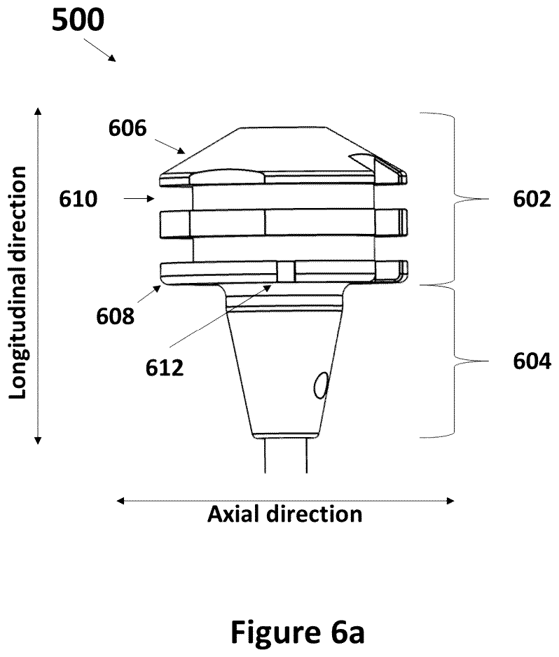

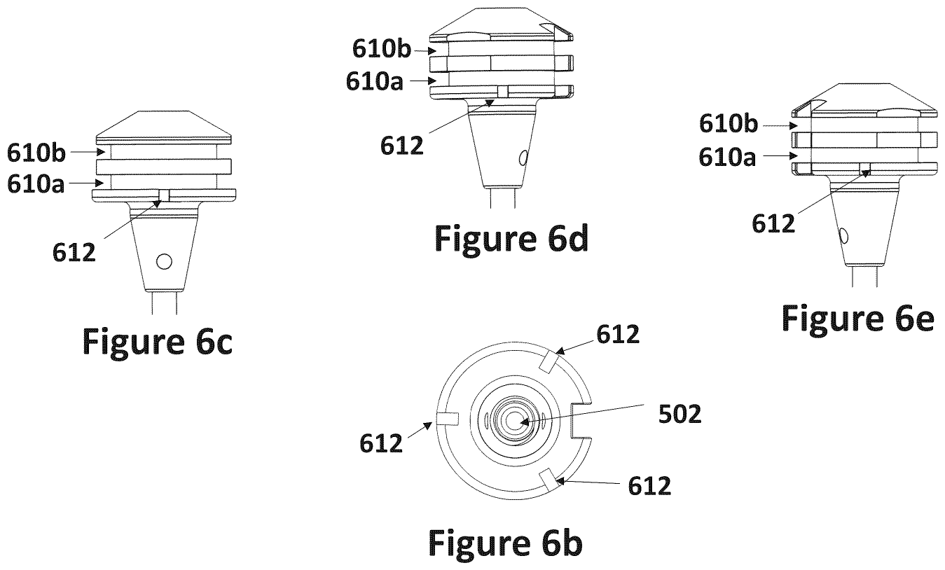

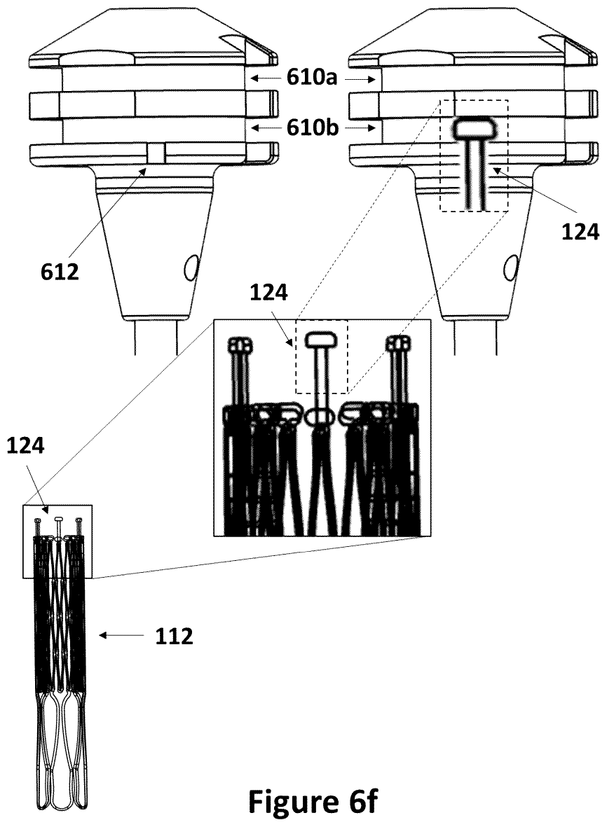

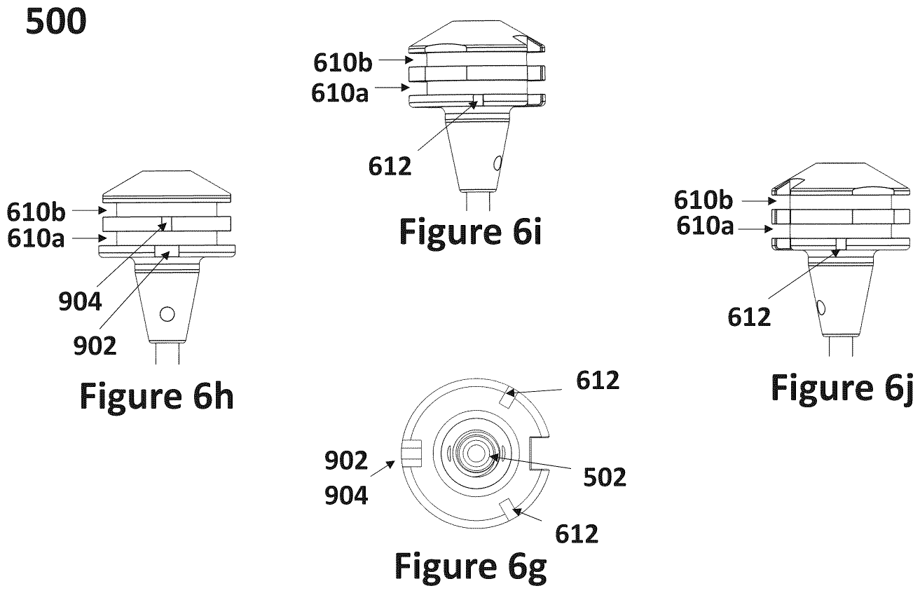

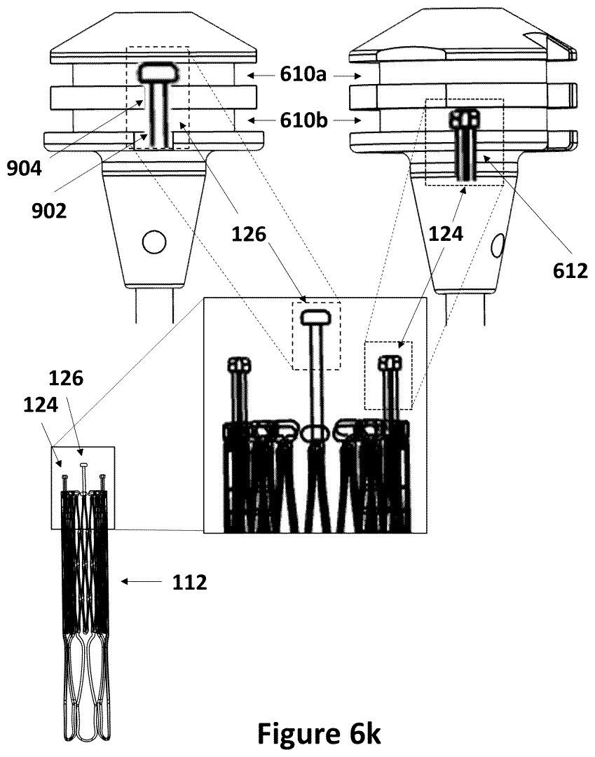

Referring now to FIGS. 6a-k, illustrating exemplary embodiments of the implant leg holder 500, according to some embodiments of the invention. Referring now to FIG. 6a, illustrating an exemplary configuration of a part of the implant leg holder, according to some embodiments of the invention. In some embodiments, the implant leg holder comprises a head 602, located proximally, and a neck 604, located distally. In some embodiments, the head 602 of the implant leg holder 500 comprises the orifice 502, as disclosed above, which is configured to be connected with the connector and therefore to the elongated body 108. In some embodiments, between the upper proximal part 606 of the head 602 and the lower distal part 608 of the head 602, there are one or more grooves 610 surrounding the perimeter of the head 602 in the general axial direction of the head 602, referred hereinafter as axial grooves. In some embodiments, the head further comprises one or more groves 612 in the general longitudinal direction of the head 602, referred hereinafter as longitudinal grooves.

In some embodiments, both the axial grooves and the longitudinal grooves are configured to receive and hold at least a part of one or more legs 124/126 of the implant 112. In FIGS. 6a-k, as a matter of example, the implant leg holder comprises 2 axial grooves (610a, 610b). In some embodiments, there can optionally be one groove, or three grooves, depending on the type of implant that is used. In some embodiments, longitudinal grooves 612 are configured to receive at least a part of an implant 112 arriving from a general distal direction (see below Exemplary preparation procedure for specific information). In some embodiments, during the preparation of the implant 112 for the implantation procedure, the user inserts the legs 124/126 in the longitudinal grooves 612, and the legs are kept in place by the combination of the axial 612 and longitudinal (610, 610a, 610b) grooves.

In some embodiments, the implant leg holder 500 comprises as many axial grooves 612 as the number of legs 124 in the implant 112. In some embodiments, the implant leg holder 500 comprises multiple axial grooves 612 configured to allow the use of the same implant leg holder 500 but with different implants having different number of legs.

Referring now to FIGS. 6b-e, illustrating an embodiment where the implant leg holder 500 comprises identical axial grooves 612, according to some embodiments of the invention. In some embodiments, the implant leg holder 500 comprises axial grooves 612 which are identical to each other. FIG. 6b illustrates a top view of the head 602 of implant leg holder 500 showing the orifice 502 having internal threaded walls and three identical axial grooves 612. FIGS. 6c-e illustrate side views showing the location of each identical axial groove 612 on the head 602 of implant leg holder 500. In some embodiments, where the axial grooves 612 are identical to each other, only implants having legs having the same length can be used.

Referring now to FIG. 6f, illustrating how an implant leg 124 is reversibly attached to the implant leg holder 500, according to some embodiments of the invention. FIG. 6f shows an implant 112 having legs 124 of the same length. In some embodiments, the legs are inserted in longitudinal grooves 612 and held inside axial groove 610b.

Referring now to FIGS. 6g-j, illustrating an embodiment where the implant leg holder 500 comprises two identical axial grooves 612 and another two different axial grooves, according to some embodiments of the invention. In some embodiments, the implant leg holder 500 comprises one or more identical axial grooves 612 configured to receive one or more legs 124 of an implant having the same length. In some embodiments, the implant leg holder 500 also comprises one or more axial grooves 902/904, which are different from axial grooves 612, and configured to receive one or more legs 126 of an implant having different length from the first ones. FIG. 6g illustrates a top view of the head 602 of implant leg holder 500 showing the orifice 502 having internal threaded walls and two identical axial grooves 612 and two different axial grooves 902, 904. FIGS. 6i-j illustrate side views showing the location of each of the identical axial grooves 612 on the head 602 of implant leg holder 500. FIG. 6h illustrates a side view showing two axial grooves 902 and 904, where groove 902 is a lower axial groove which, in this example, is wider than axial grooves 612, but at the same height as axial grooves 612. Groove 904 is located higher than groove 902 which, in this example, comprises the same width as axial grooves 612, but is located at a higher location than axial grooves 612. In some embodiments, different types of legs 124/126 and/or different types of axial grooves 612, 902, 904 are used to provide a directional configuration of the mounting of the implant 112 on the implant leg holder 500 (see below).

Referring now to FIG. 6k, illustrating how an implant 112 having two short legs 124 and one long leg 126 are reversibly attached to the implant leg holder 500, according to some embodiments of the invention. FIG. 6k shows an implant 112 having two short legs 124 and one long leg 126. In some embodiments, the short legs are inserted in longitudinal grooves 612 and held inside axial groove 610b, as shown on the right side of the Figure, while the long leg 126 is inserted in longitudinal groove 902, passing through axial groove 610b, inserted also in longitudinal groove 904 (located higher than longitudinal groove 902) and held inside axial groove 610a, as shown for example in the left side of the Figure.

### Exemplary Implant Capsule/Enclosure 406

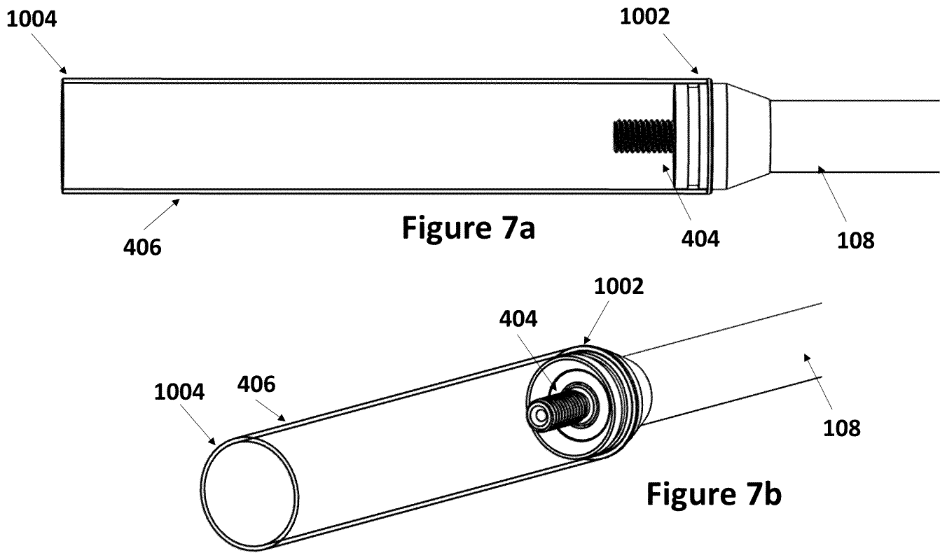

Referring now to FIGS. 7a-b, illustrating an exemplary implant capsule/enclosure 406, according to some embodiments of the invention. FIG. 7a illustrates a side view of an exemplary implant capsule/enclosure 406, while FIG. 7b illustrates a perspective view of an exemplary implant capsule/enclosure 406. As mentioned above, at the distal end of the internal tube of the elongated body 108 there is a connector, optionally configured as a “male”-threaded extension, and configured to be used to connect with the head 602 of the implant holder 402. The “male-female”-screwing threaded configuration is just an example, other configurations of connectors can be used to connect the implant holder 402 and the internal tube of the elongated body 108, for example a push-pull connectors, bayonet (or reverse bayonet) coupling connectors, just to name a few. In some embodiments, the implant holder 402 is covered by the implant capsule/enclosure 406. In some embodiments, the proximal end 1002 of the implant capsule/enclosure 406 is irreversible connected to the external tube 302 of the elongated body 108. In some embodiments, the implant capsule/enclosure is made of transparent materials, such as Nylon/Pebax/PEEK. In some embodiments, the length of the implant capsule/enclosure 406 is as long as the implant holder 402, which is as long as the crimped configuration of the implant 112. In some embodiments, the length of the implant capsule/enclosure 406 is from about 45 mm to about 60 mm; optionally from about 40 mm to about 70 mm; optionally from about 30 mm to about 80 mm. In some embodiments, the internal diameter of the implant capsule/enclosure 406 is approximately the same or bigger as the implant holder 402 comprising the crimped configuration of the implant 112. In some embodiments, the internal diameter of the implant capsule/enclosure 406 is from about 9 mm to about 10 mm; optionally from about 8 mm to about 12 mm; optionally from about 7 mm to about 15 mm; for example 9 mm, 9.7 mm, 10.5 mm. In some embodiments, the capsule is transparent. In some embodiments, a potential advantage of having a transparent capsule is that it allows to monitor the loading process of the implant into the capsule.

### Exemplary Distal Tip 400

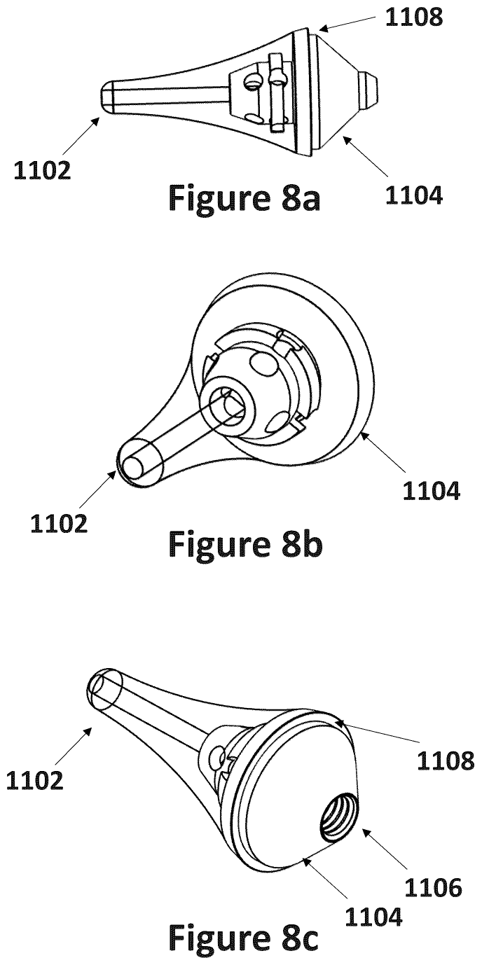

Referring now To FIGS. 8a-c, illustrating different angles of an exemplary distal tip 400, according to some embodiments of the invention. In some embodiments, the distal tip 400 comprises the following parts: an atraumatic distal end 1102 and a distal tip base 1104. In some embodiments, the distal tip base 1104 comprises an orifice 1106 having internal threaded walls and configured to be connected with the threaded adaptor 506 located at the distal end of the implant holder 402. In some embodiments, other technologies are used, for example snap attachments, etc.. In some embodiments, the distal tip base 1104 is made of one or more of PEEK/Pebax/Stainless steel. In some embodiments, the atraumatic distal end 1102 is made of one or more of Silicone/Pebax. In some embodiments, the distal tip base 1104 comprises a groove 1108 configured to match the distal end 1004 of the implant capsule/enclosure 406 and enable a tight, optionally hermetic, closure of the internal space of the implant capsule/enclosure 406.

### Exemplary Steering Movements of the Distal End of the Elongated Body

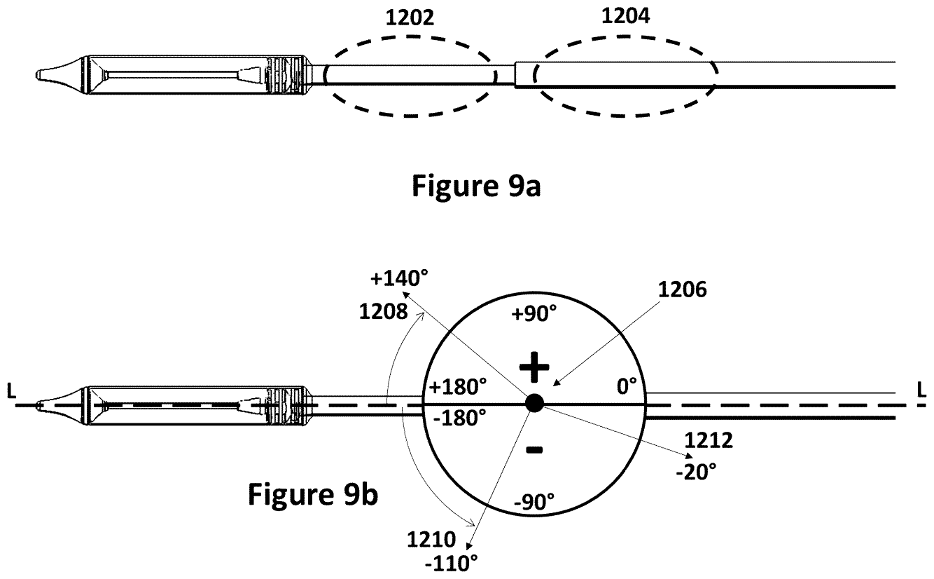

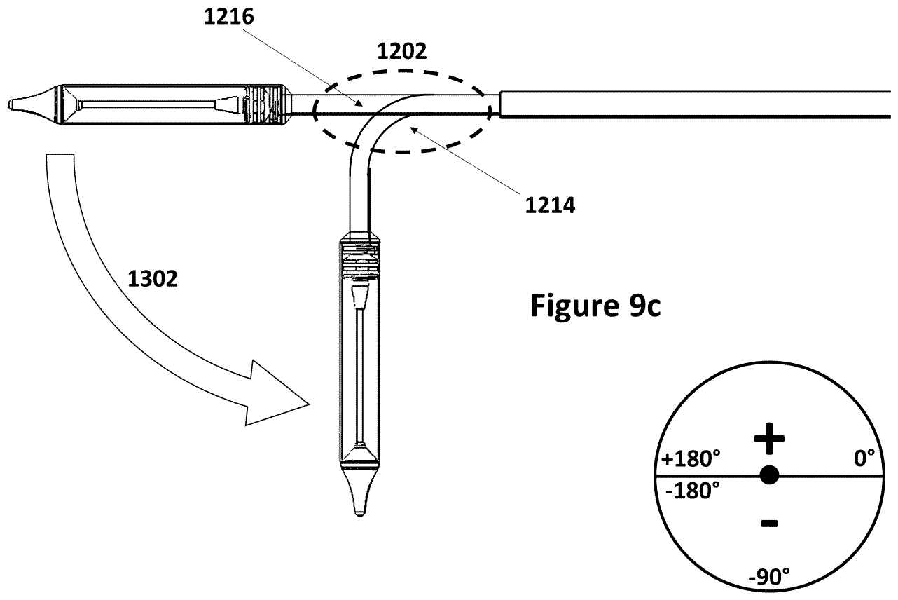

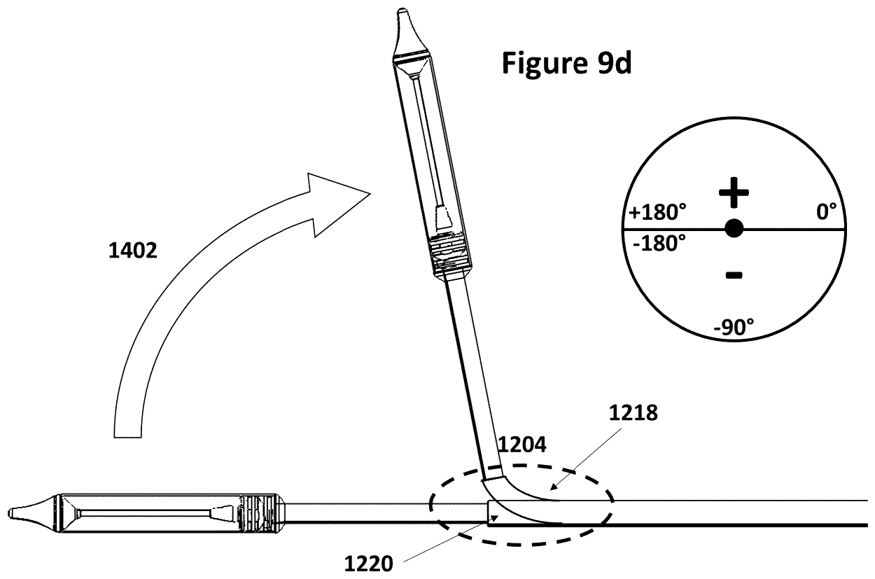

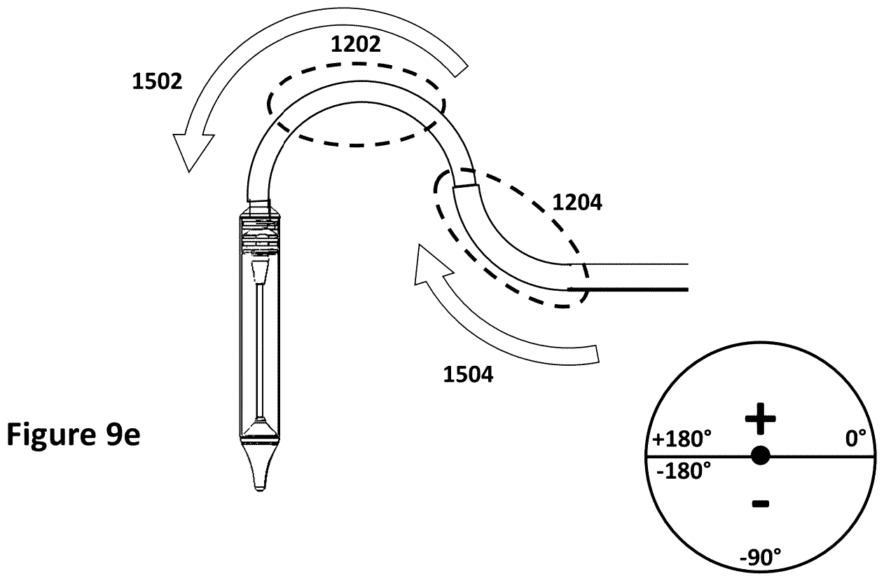

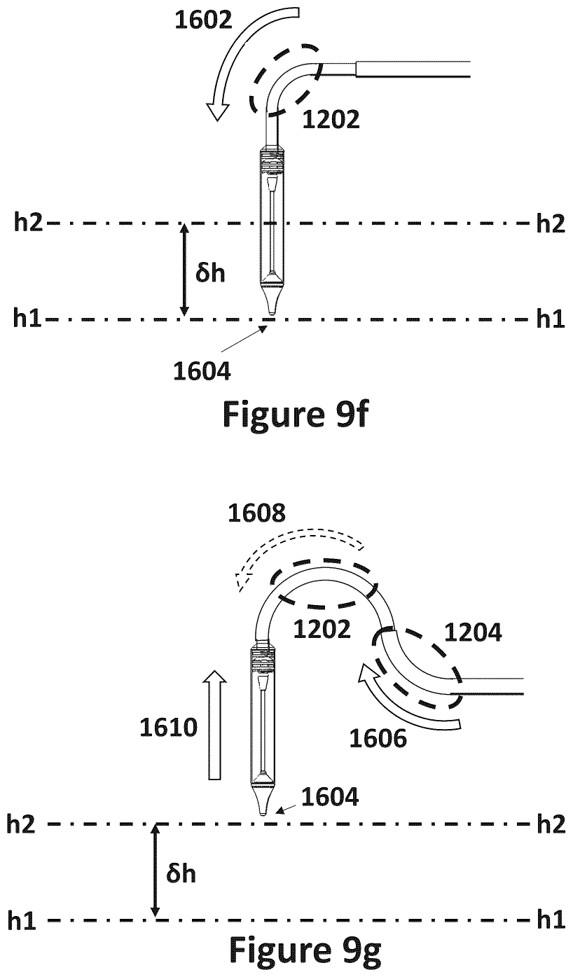

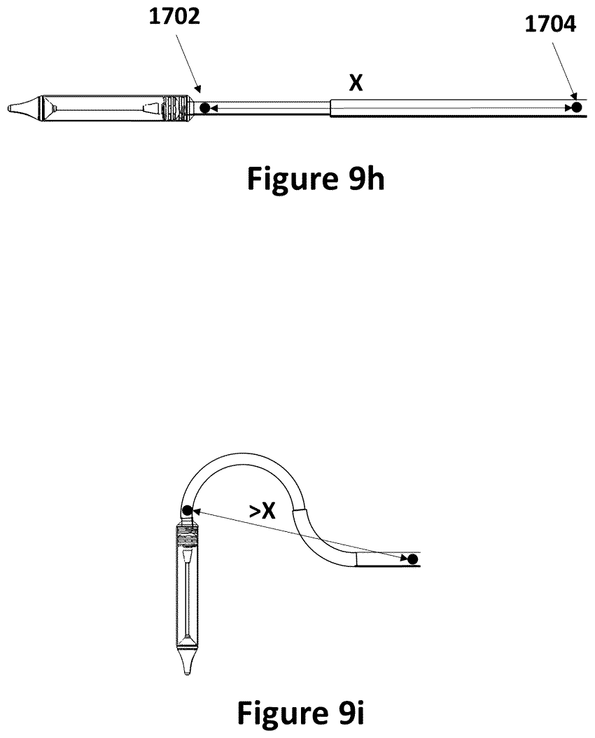

Referring now to FIGS. 9a-c, illustrating exemplary steering movements of the distal end of the elongated body, according to some embodiments of the invention. In some embodiments, the distal end of the elongated body 108 comprises one or more unidirectional steering zones, meaning, zones in the elongated body 108 where the elongated body is bended to one direction. In some embodiments, the elongated body comprises two steering zones, one steering zone 1202 in close proximity to the implant housing/operational distal end 110 and another steering zone 1204 proximally to steering point 1202. In some embodiments, one steering zone bends to one direction while the other steering zone steers to an opposite and/or different direction. Referring now to FIG. 9b, showing a schematic representation of a distal part of the device together with a virtual angle gauge, which will help explaining the direction of the movement of the elongated body 106 with respect of a general longitudinal line LL, when observing the device from a side point of view. Black dot 1206 represents the general flexing and/or bending point on the device. For example, if the distal end of the device bends up, as shown by arrow 1208, it means that the device moves from 180° to an angle 140°, with respect to the 0°, as shown by the angle gauge. Another example, if the device bends down, as shown by arrow 1210, it means that the device moves from 180° to an angle −110°, with respect to the 0°, as shown by the angle gauge. One last example, if the device bends down, as shown by arrow 1212, it means that the device moves from 180° to an angle −20°, with respect to the 0°, as shown by the angle gauge.

Referring now to FIG. 9c, illustrating an exemplary bending movement in zone 1202, according to some embodiments of the invention. In some embodiments, distal zone 1202 bends down following the arrow 1302. In some embodiments, the bending angle is from about 180° (or −180°) to about −0° (meaning the distal end is turned so as to look back at the elongated body), optionally from about 1800 (or −180°) to about −20°, optionally from about 1800 (or −180°) to about −50°. In some embodiments, the movement of the distal zone 1202 is actuated by an actuation mechanism in the handle 106, for example by actuating control knob 206. In some embodiments, when the distal zone 1202 is bent, it generates an internal side 1214 of the distal bending zone and an external side 1216 of the distal bending zone, as shown by arrows in FIG. 9c.

Referring now to FIG. 9d, illustrating an exemplary bending movement in zone 1204, according to some embodiments of the invention. In some embodiments, proximal zone 1204 bends up following the arrow 1402. In some embodiments, the bending angle is from about 1800 (or −180°) to about 0° (meaning the distal end is turned so as to look back at the elongated body), optionally from about 1800 (or −180°) to about 20°, optionally from about 1800 (or −180°) to about 50°. In some embodiments, the movement of the proximal zone 1204 is actuated by an actuation mechanism in the handle 106, for example by actuating control knob 208.

In some embodiments, when the proximal zone 1204 is bent, it generates an internal side 1218 of the proximal bending zone and an external side 1220 of the proximal bending zone, as shown by arrows in FIG. 9d.

Referring now to FIG. 9e, illustrating an exemplary bending movement in both zone 1202 and zone 1204, according to some embodiments of the invention. In some embodiments, the combination of the movements of the distal zone 1202 (shown by arrow 1502) and the proximal zone 1204 (shown by arrow 1504) provides a device that can bend as shown in FIG. 9e.

Referring now to FIGS. 9f and 9g, illustrating an exemplary combination of movements of the distal end of the device, according to some embodiments of the invention. In some embodiments, the device is first bent as shown in FIG. 9f, by actuating control knob 206 for example, and causing the distal zone 1202 to bend down to an exemplary angle of −90°, following arrow 1602. At the end of the bending actuation, the distal end of the device (marked as 1604) “faces down” at a relative height h1. Then, proximal zone 1204 is bent up, by actuating control knob 208 for example, following arrow 1606, while continuing actuating control knob 206, therefore continuing bending down distal zone 1202, following dashed-arrow 1608, thereby keeping the distal end of the device “facing down”. At the end of the bending actuation, the distal end of the device (marked as 1604) still “faces down”, but this time at a relative height h2. The different between relative height h1 and h2 is denoted as δh. In some embodiments, the combination of movements enables the device to first “face down” after a bending of about −90°, and then “move up”, while still “face down”.

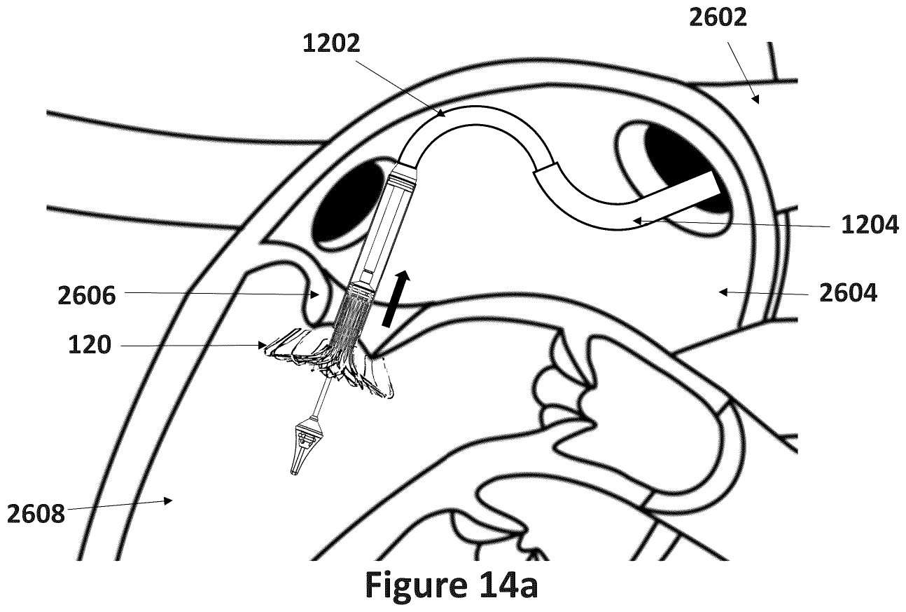

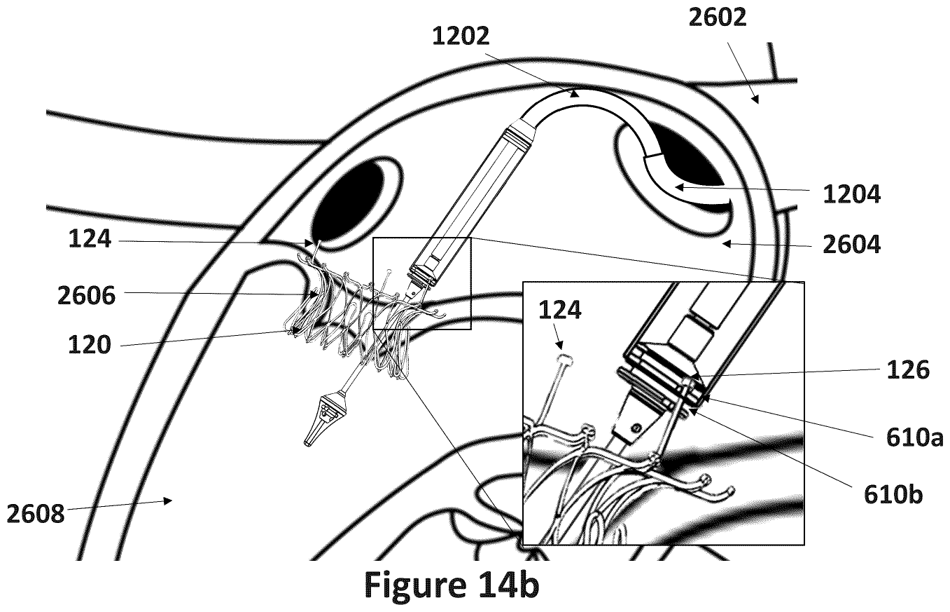

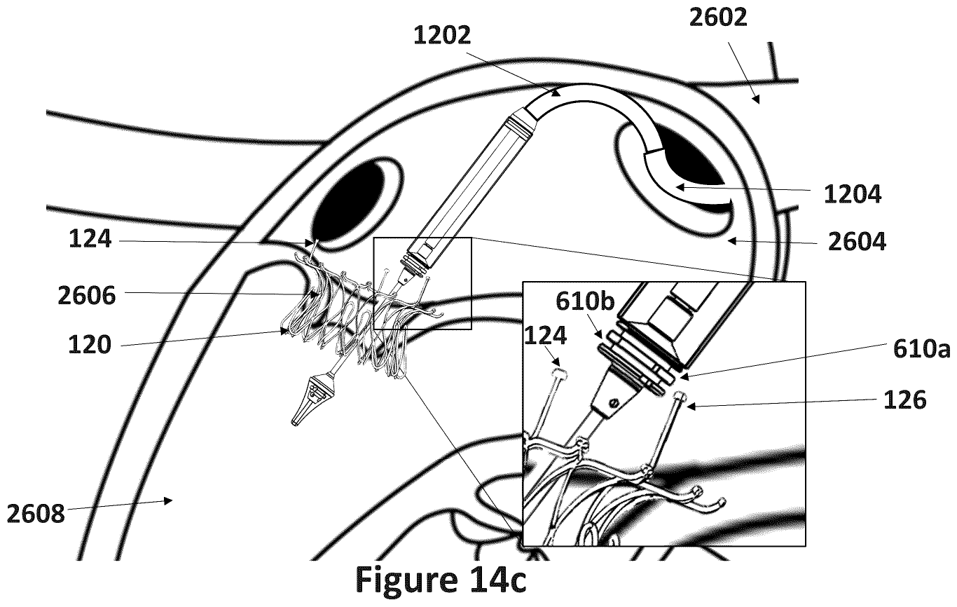

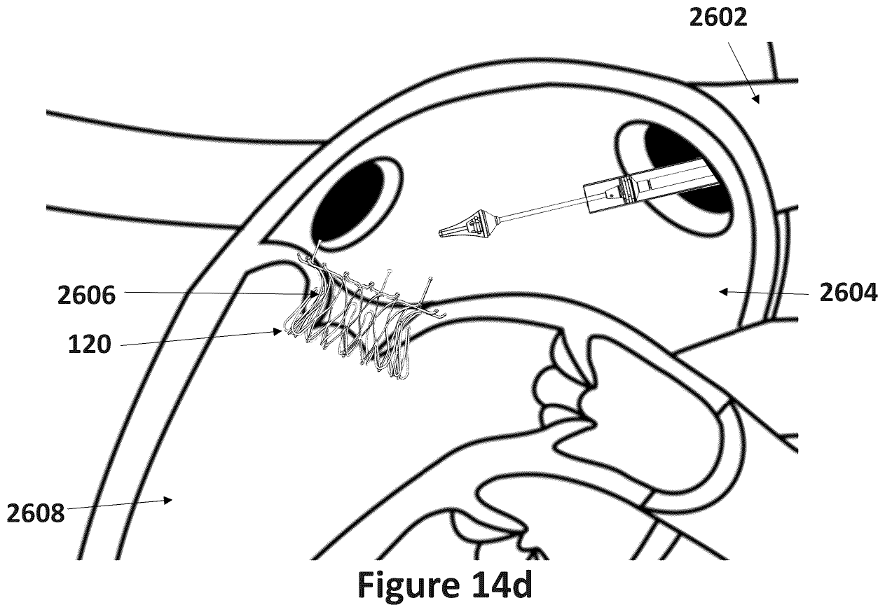

In some embodiments, the combination of the movements of both bending locations allow the specific movements required of the delivery device inside the heart for the delivery of the heart implant in the right location (see FIGS. 14a-14g and 15a-15d and related explanations).

Referring now to FIGS. 9h and 9i, showing a schematic representations of definition of movements of the delivery device, according to some embodiments of the invention. In some embodiments, a way to explain the movements of the delivery system is by imagining two virtual points (1702 and 1704) on the shaft of the delivery device, as shown for example in FIG. 9h. In some embodiments, a virtual chord can be established between points 1702 and 1704, which will comprise a length (x). In some embodiments, the bending actions of the shaft at the two location causes a shortening of the virtual chord, which will comprise a shorter length (>x), as shown for example in FIG. 9i. In some embodiments, the manipulation of the shaft by bending it at one or more locations is defined as the actions required to shorten a virtual chord generated by two virtual points located on the shaft of the delivery device.

### Exemplary Steering Mechanisms

In some embodiments, an exemplary steering mechanisms comprises the introduction of rings and cables in the manufacturing process of the tubes. In some embodiments, pulling and/or pushing and/or releasing a cable (for example by actuating a knob in the handle) causes the bending of the tube at the location where the ring is located in the specific tube. In some embodiments, these mechanisms of steering used are those known in the art, and variations of steering mechanisms are included in the scope of the invention.

### Exemplary Proximal/Distal Movements of the Elongated Body 108

In some embodiments, external tube 302 is configured to move proximally or distally while internal tube is kept still. In some embodiments, the proximal/distal movement is controlled, for example, by control knob 210 located at the handle. In some embodiments, rotating control knob 210 to one direction causes the external tube 302 to move forward (e.g. distally), while rotating control knob 210 to the other direction causes external tube 302 to move backwards (e.g. proximally.

In some embodiments, the distal/proximal movements causes the external tube 302 to move distally or proximally, and since the implant capsule/enclosure 406 is irreversibly connected to the external tube 302, when the external tube 302 is moved proximally, while keeping the internal tube steady, the implant holder is exposed, as will be further explained below in the “Exemplary Implantation Procedure”.

### Exemplary Quick Release Knob 212

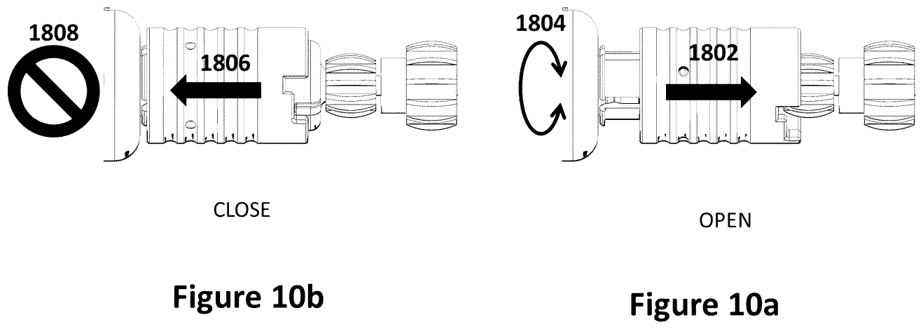

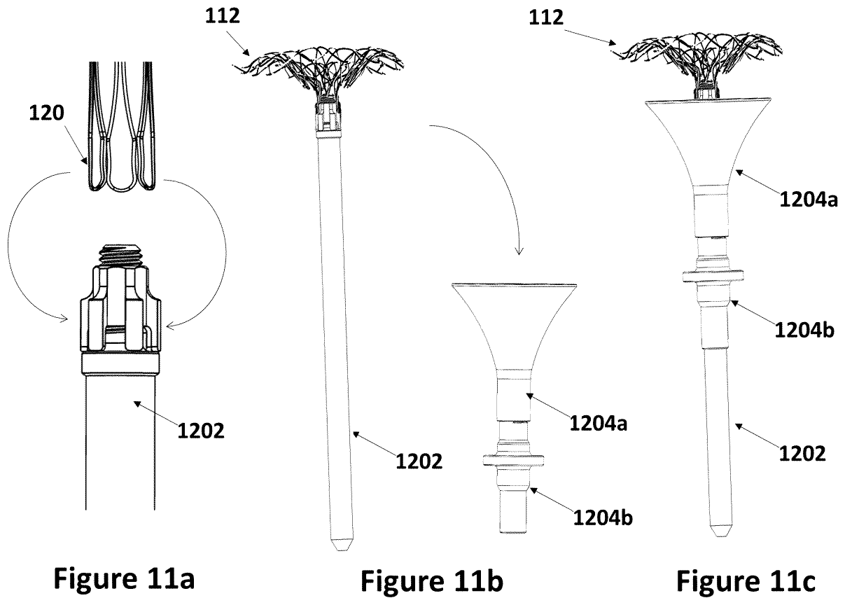

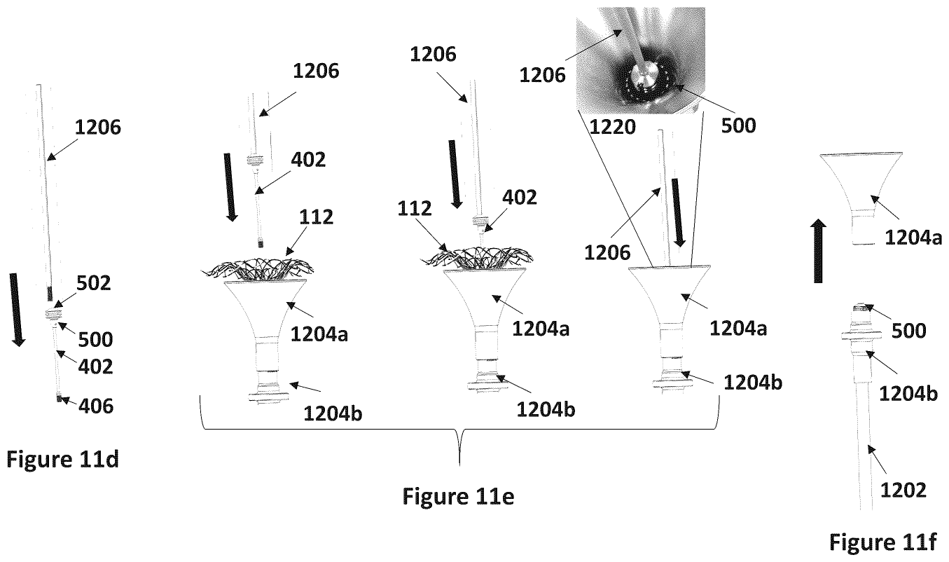

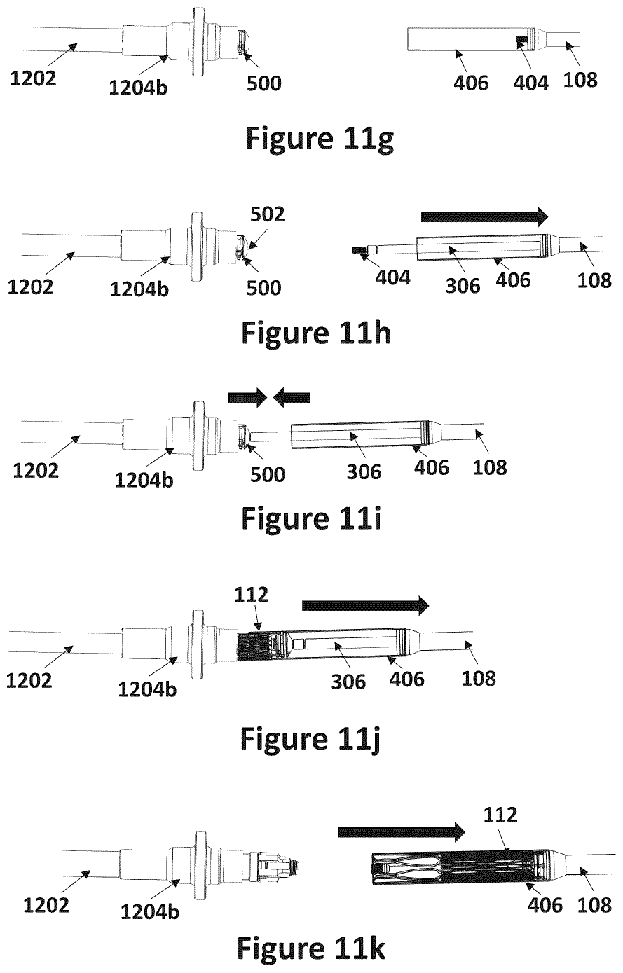

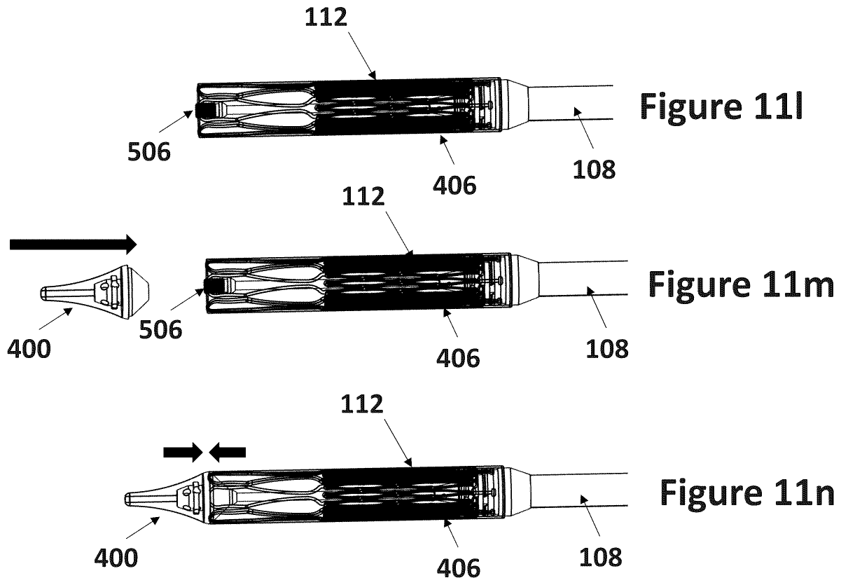

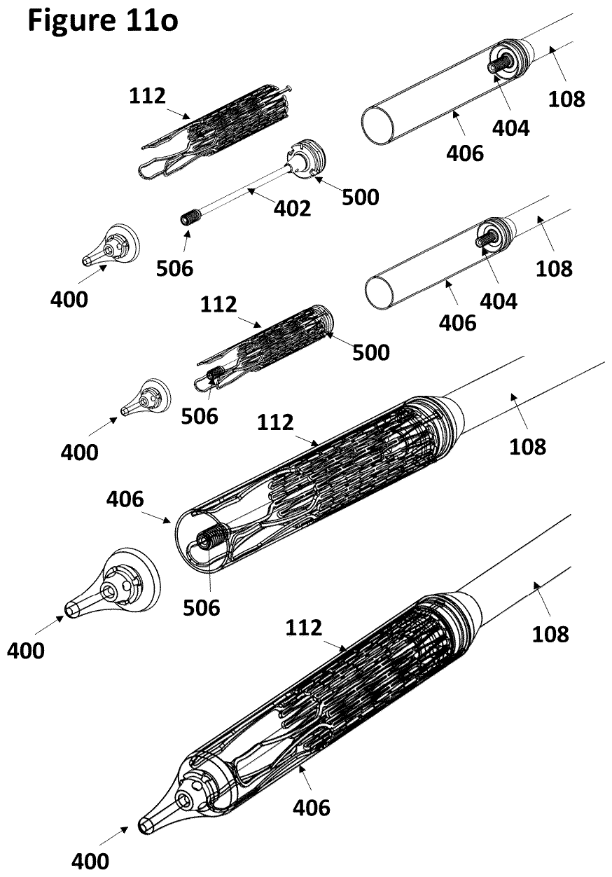

Referring now to FIGS. 10a-b, illustrating an exemplary quick release knob 212, according to some embodiments of the present invention. In some embodiments, internal tube ends at the proximal end of the handle 106. In some embodiments, also located at the proximal end of the handle 106, there is a quick release knob 212, connected to the proximal end of the internal tube. In some embodiments, opening the quick release knob 212, for example by pulling it proximally (see arrow 1802), as shown for example in FIG. 10a, allows the user to easily rotate the internal tube either right or left along the longitudinal axis of the internal tube, as marked by circular arrow 1804. In some embodiments, closing the quick release knob 212, for example by pulling it distally (see arrow 1806), as shown for example in FIG. 11b, allows the user to lock the internal tube so it cannot rotate neither right nor left along the longitudinal axis of the internal tube, as marked by the “no” symbol 1808. In some embodiments, the rotation of the internal tube does not rotate the external tube 302 and/or the implant capsule/enclosure 406. In some embodiments, the exemplary quick release knob allows the user to move the internal tube of the delivery device distally and/or proximally without moving the external tube. In some embodiments, a potential advantage of this mechanism is that it allows the user to move the implant holder (with the implant) distally and proximally without the need to move the external tube and/or the implant capsule/enclosure. In some embodiments, this is helpful during the deployment of the implant as it allows the user to quickly reinsert the implant into the capsule/enclosure in case is required.

### Exemplary Preparation Procedure

Referring now to FIGS. 11a-o, illustrating an exemplary preparation procedure of the system according to some embodiments of the invention. In some embodiments, the preparation of the system commences by reversibly attaching the anchors 120 into an anchor-holder rod 1202. In some embodiments, the attachment of the anchors 120 onto the anchor-holder rod 1202 is performed by pulling down the anchors 120, thereby positioning the anchors 120 in an open configuration, as can be seen for example in FIG. 11a. In FIG. 11b it can be seen the anchor-holder rod 1202 with the implant 112 attached to its distal end. The proximal end of the anchor-holder rod 1202 is inserted into a two part funnel 1204a/1204b, where the top part of the funnel is numbered 1204a and the bottom part is numbered 1204b. FIG. 11c shows the anchor-holder rod 1202 with the implant 112 inside the two part funnel 1204a/1204b. Then, a helper rod 1206 is inserted into the orifice 502 of the implant leg holder 500 of the implant holder 40, as shown for example in Figure lid. Then, the threaded adaptor 506 is screwed into the head of the anchor-holder rod 1202. Then, the anchor-holder rod 1202 is further pulled down so that the rest of the implant 112 will be in contact with the internal wall of the upper part 1204a of the funnel and close up on himself while being pulled down, as shown for example in FIG. 11e. FIG. 11e also show, in square 1220, that once the implant leg holder 500 arrives at the narrowest part of the upper part 1204a of the funnel, the legs 124 of the implant 112 coincide with the implant leg holder 500. At this point, the user inserts the legs 124 into the dedicated grooves 612, as shown for example in FIG. 6b. Once the insertion of the legs 124 into the grooves 612 on the implant leg holder 500 are finished, the user detaches the helper rod 1206 and separates between the upper part 1204a and the lower part 1204b of the funnel, as shown for example in FIG. 11f. At this point the implant 112 is crimped and ready to be inserted in the delivery device 100. The user brings the crimped implant 112, held by the anchor-holder rod 1202 and the lower part 1204b of the funnel in proximity to the distal end of the delivery device 100, as shown for example in FIG. 11g. The user then rotates, for example, control knob 210 to cause the external tube 302 (together with the implant capsule/enclosure 406) to move backwards (proximally) thereby exposing the internal tube and the connector, as shown for example in FIG. 11h. Then the user connects between the connector, located at the distal end of the internal tube and the orifice 502 in the implant leg holder 500 of the implant holder 402, as shown for example in FIG. 11i Then the user rotates knob 210 to the other direction to cause the external tube 302 (together with the implant capsule/enclosure 406) to move forward (distally), thereby covering the implant holder 402 comprising the implant 112 with the implant capsule/enclosure 406, as shown for example in FIG. 11j. Once the implant holder 402 comprising the implant 112 are located completely inside the implant capsule/enclosure 406, the user disconnects the anchors 120 and the threaded adaptor 506 from the anchor-holder rod 1202, thereby releasing the implant holder 402 comprising the implant 112, which are now inside the implant capsule/enclosure 406, as shown for example in Figure ilk. Once the implant holder 402 comprising the implant 112 are inside the implant capsule/enclosure 406, as shown for example in FIG. 11l, the user brings the distal tip 400, as shown for example in FIG. 11m. Lastly, the user attaches the threaded adaptor 506 of the implant holder 402 with the orifice 1106 of the distal tip base 1104 located in the distal tip 400.

Referring now to FIG. 1o, illustrating the different parts of the delivery system 100 in an exploded perspective view, and the way they connect to each other, provided to facilitate the understanding of the delivery system 100, according to some embodiments of the invention.

### Exemplary Implantation Procedure

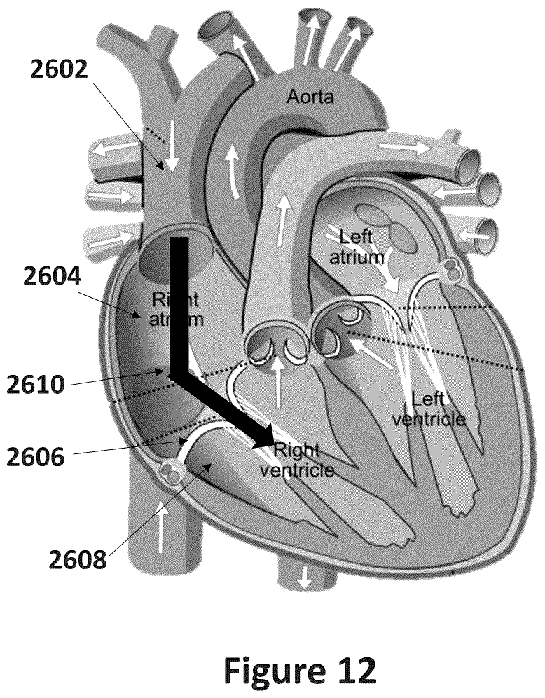

Referring now to FIG. 12, illustrating a schematic representation of the path that the delivery device performs during the implantation procedure, according to some embodiments of the invention. In some embodiments, during the implantation procedure, the delivery device is inserted, for example, via the jugular vein 2602 of the patient and brought directly into the right atrium 2604 of the heart. In order to implant the device at the tricuspid valve 2606, between the right atrium 2604 and the right ventricle 2608, the delivery device must perform a turn, as shown for example by the black arrow 2610 in FIG. 12. It is known that this non-straight access to the tricuspid valve 2606 increases the difficulties of implanting the device at the right location during implantation. A potential advantage of the delivery system 100 disclosed herein is that the delivery system 100 of the invention allows the user to overcome the anatomical difficulties of the heart by providing an easy to maneuver delivery device which allows not only easy access to the right location but also enables the user to perform corrections in the location of the device once deployed.

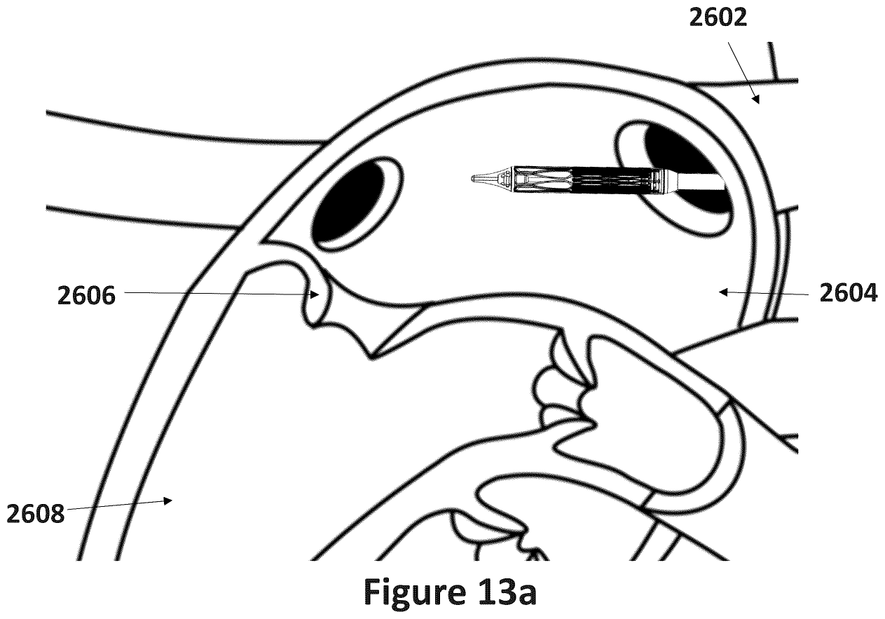

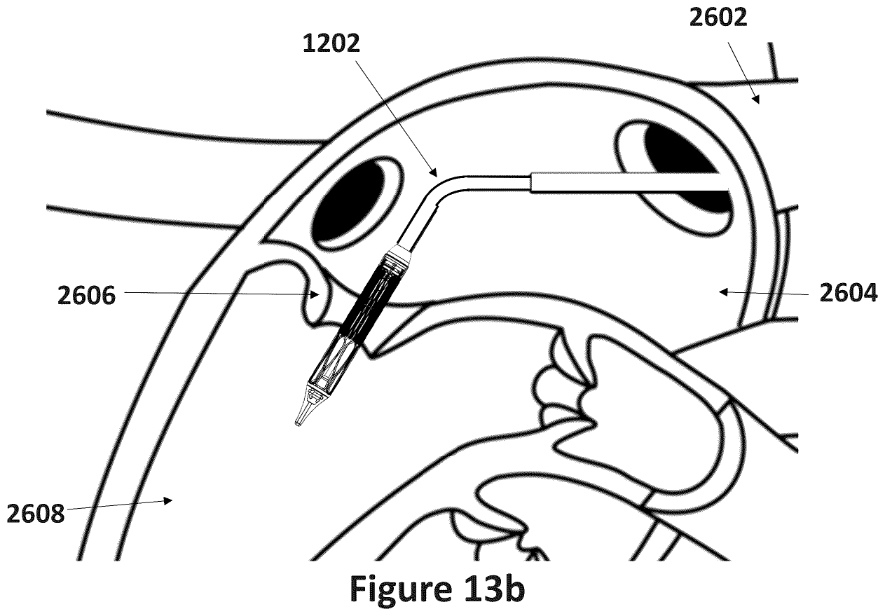

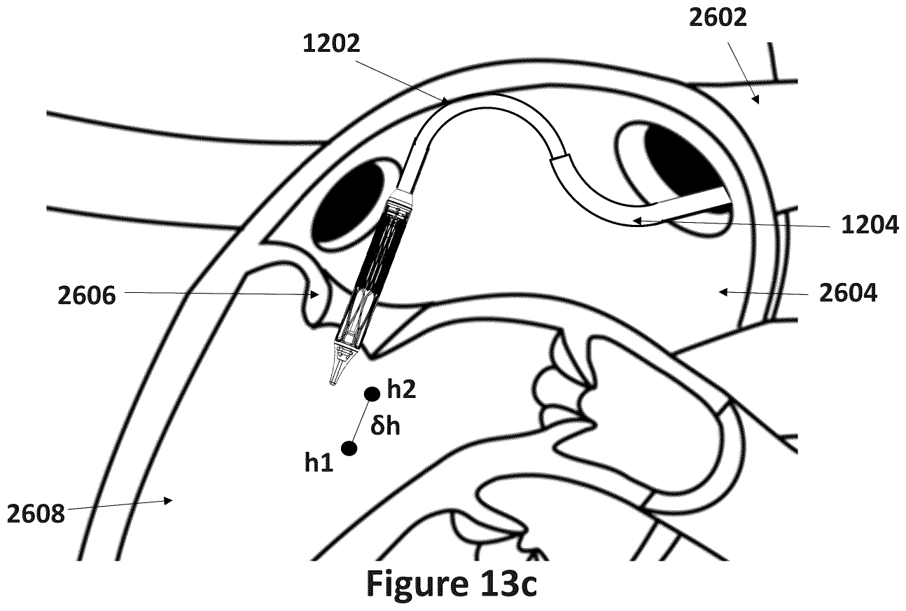

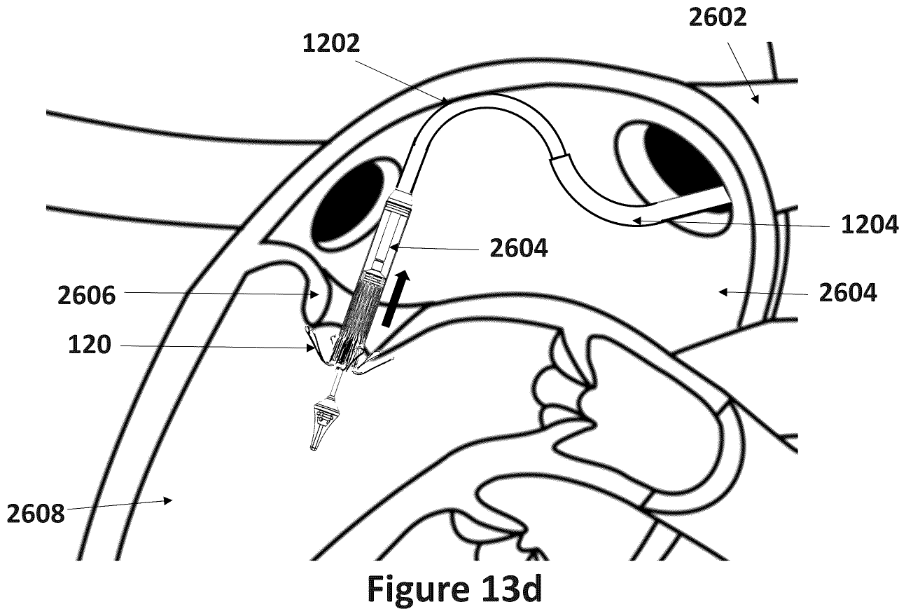

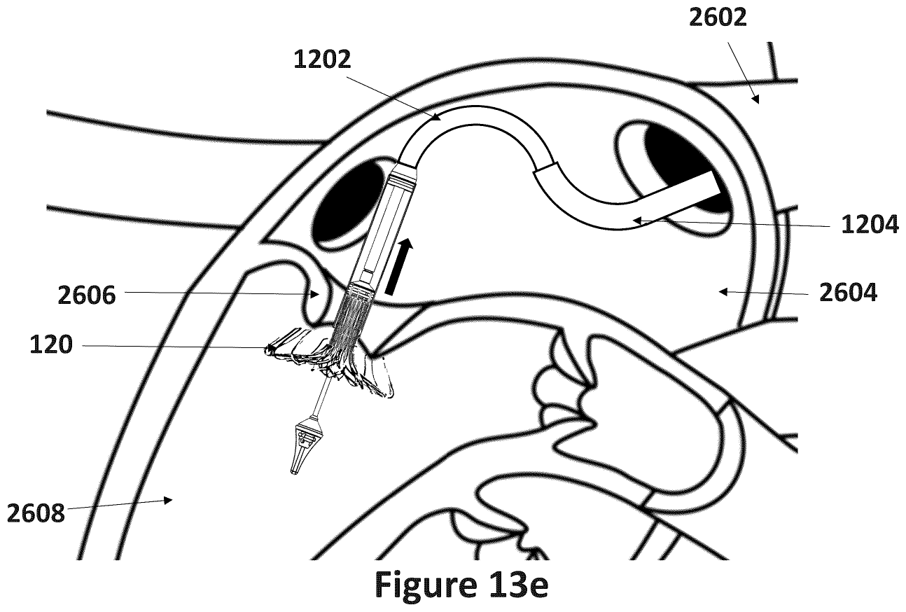

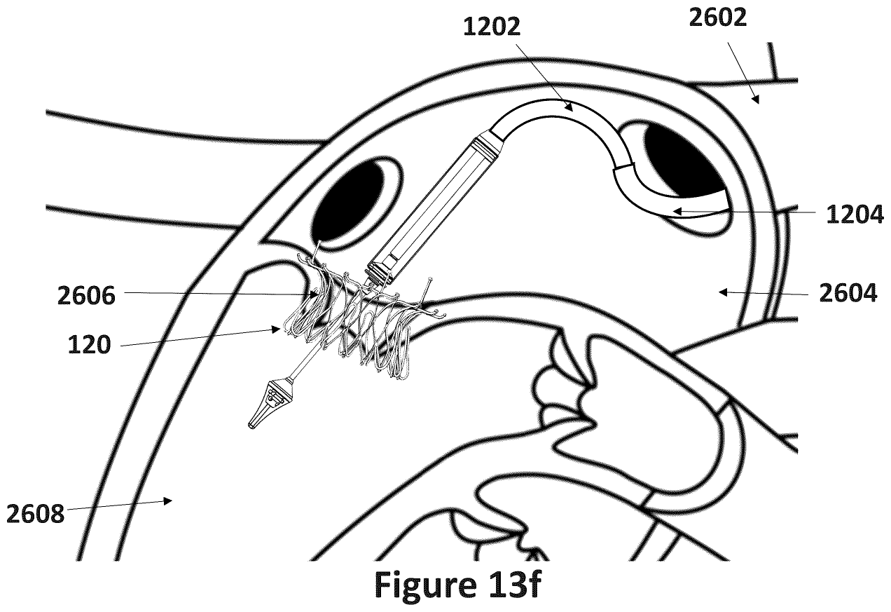

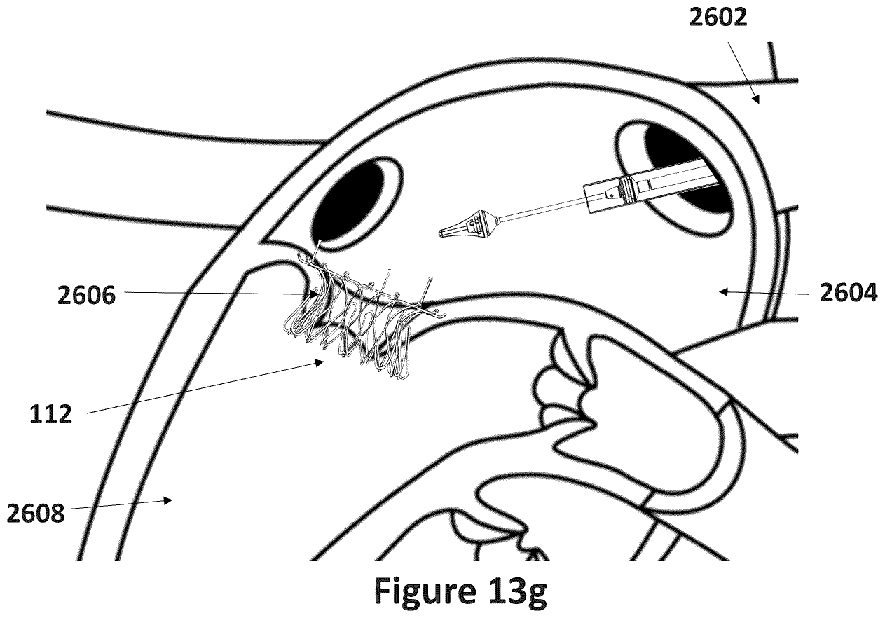

Referring now to FIGS. 13a-g, illustrating an exemplary delivery method of an implant having legs with the same length, according to some embodiments of the invention. In some embodiments, the device is brought into the right atrium 2604, as shown for example in FIG. 13a. Then the user actuates the bending of the distal zone 1202 towards the tricuspid valve 2606 and through it towards the right ventricle 2608, as shown for example id FIG. 13b. Then the user actuates the bending of the proximal zone 1204 thereby pulling the distal end of the delivery device upwards and centering it in the middle of the tricuspid valve 2606, moving upwards a distance δh from h1 to h2 (as explained in FIGS. 9g-h), as shown for example in FIG. 13c and the actuation of the bending of the device as shown for example in FIGS. 9a-f. Once in place, the user actuates the external tube 302 to move backwards (see black arrow), while keeping the internal tube in place, and thereby beginning to release the anchors 120, which begin to fold back behind the tricuspid valve 2606, as shown for example in FIG. 13d. The user continues to actuate the external tube 302 to move backwards (see black arrow), while still keeping the internal tube in place, and thereby finishing to release the anchors 120, which are now completely folded back behind the tricuspid valve 2606, in the right ventricle 2608, as shown for example in FIG. 13e. In some embodiments, when the implant 112 comprises three identical legs 124 in length, as shown for example in FIGS. 1b-c and FIG. 6f, once the legs 124 are released from the implant leg holder 500, in the right atrium 2604, the implant 112 returns to its open configuration (as shown for example in FIG. 1b) and stays in place on the tricuspid valve 2606, between the right atrium 2604 and the right ventricle 2608, as shown for example in FIG. 13f. Then, the delivery device can be extracted from the heart and the patient, while leaving the implant 112 in place, as shown for example in FIG. 13g.

Referring now to FIGS. 14a-d, illustrating an exemplary delivery method of an implant having legs with different lengths, according to some embodiments of the invention. FIG. 14a is the same as FIG. 13e, where the user continues to actuate the external tube 302 to move backwards (see black arrow), while still keeping the internal tube in place, and thereby finishing to release the anchors 120, which are now completely folded back behind the tricuspid valve 2606, in the right ventricle 2608. In this embodiment, the implant comprises two short legs 124 attached in lower grooves 612 and axial groove 610b and one long leg attached in higher groove 904 and axial groove 610a (as explained in FIG. 6k). Therefore, the user continues to actuate the external tube 302 to move backwards while still keeping the internal tube in place, until axial groove 610b is outside the implant capsule/enclosure 406, thereby releasing the short legs 124 while still holding the long leg 126 in axial groove 610a inside the implant capsule/enclosure 406—see zoom-in square, as shown for example in FIG. 14b. In some embodiments, due to the anatomy of the heart, the direction of the implant in the crimped configuration, in relation to the direction of the tricuspid valve 2606, are usually unaligned. In some embodiments, due to the configuration provided by the long leg 126 and the axial groove 610a inside the implant capsule/enclosure 406, once the short legs 124 are released, the partially deployed implant aligns itself to the tricuspid valve 2606, while still holding the long leg 126.

In some embodiments, the location of the long leg 126 and the axial groove 610a inside the implant capsule/enclosure 406, in relation to the distal bending zone of the delivery device, are maneuvered until the long leg 126 and the axial groove 610a are aligned with the internal side 1214 of the distal bending zone 1202 (see FIG. 9c). In some embodiments, as mentioned above, the user can actuate, for example, the quick release to allow the rotation of the internal tube without moving the external tube. In some embodiments, once the device is bent for the delivery of the implant in the tricuspid valve 2606, the user actuates the quick release to rotate the internal tube, which also rotates the implant holder and therefore also the implant, to bring the long leg 126 and the axial groove 610a inside the implant capsule/enclosure 406 to the correct position, which is on the same side where the internal side 1214 of the distal bending zone 1202 is located. At this point, the user can actuate distal zone 1202 and/or proximal zone 1204 to move the implant 112 and perform corrections in the location of the implant 112 in the tricuspid valve 2606. The user then continues to actuate the external tube 302 to move backwards while still keeping the internal tube in place, until axial groove 610a is outside the implant capsule/enclosure 406, thereby releasing the long leg 126—see zoom-in square, thereby releasing the implant 112 from the delivery device, as shown for example in FIG. 14c. Then, the delivery device can be extracted from the heart and the patient, while leaving the implant 112 in place, as shown for example in FIG. 14d.

It is expected that during the life of a patent maturing from this application many relevant tricuspid implant valves and delivery systems thereof will be developed; the scope of the terms “implant” and/or “delivery system” are intended to include all such new technologies a priori.

As used herein with reference to quantity or value, the term “about” means “within ±20% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A delivery system for a prosthetic heart valve, comprising:
a. an elongated body comprising a proximal end, a distal end and at least two bending portions, said two bending portions having different bending directions;
b. an inner tube comprising a proximal end and a distal end, and positioned inside an outer tube;
c. an operational distal end located at said distal end of said outer tube; said operational distal end comprising:
i. an implant holder reversibly attached to said distal end of said inner tube; said implant holder comprising at least two implant leg holders, one configured for holding one of said legs in a more axial proximally position than the other;
ii. an atraumatic distal tip connected to a distal end of said implant holder; and
iii. an implant enclosure for externally enclosing said implant, proximally connected to said distal end of said outer tube, and extending until a proximal end of said distal tip;
d. a virtual chord being defined between two locations adjacent to said operational distal end; said virtual chord having a length; wherein shortening said length of said virtual chord by actuating said at least two bending portions aligns said operational distal end ±20 degrees perpendicular to an opening of said heart valve.

2. (canceled)

3. The delivery system according to claim 1, wherein said bending portions are configured to bent to a bending angle of from about 0 degrees to about 180 degrees.

4. (canceled)

5. The delivery system according to claim 1, wherein said elongated body is characterized by one or more of:
a. being long enough to reach a heart of a patient from the outside of the body of said patient using a femoral entry or a jugular entry;
b. comprising external flexible tube;
c. comprising a port to be located at an incision point in said body of said patient;
d. being configured to move along said port.

6-7. (canceled)

8. The delivery system according to claim 5, wherein said port comprises and inlet-outlet tube for inserting and removing liquids from a blood vessel.

9. (canceled)

10. The delivery system according to claim 1, wherein said inner tube is one long tube; and wherein said one long tube comprises a plurality of zones having different levels of stiffness.

11. (canceled)

12. The delivery system according to claim 1, wherein said inner tube comprises two or more parts.

13. The delivery system according to claim 1, wherein said inner tube comprises two parts, a first part extending from a handle of said delivery system to a certain location of said elongated body and a second part extending from said certain location of said elongated body to said operational distal end; and wherein one or more of the following is true:
a. said second part is covered with an envelope;
b. said first part is stiffer than said second part;
c. said first part is from about 0.1% to about 50% stiffer than said second part;
d. said second part is more flexible than said first part:
e. said second part is from about 0.1% to about 50% more flexible than said first part.

14-20. (canceled)

21. The delivery system according to claim 1, wherein said implant enclosure is sized and shaped to accommodate said prosthetic heart valve in a crimped configuration.

22. The delivery system according to claim 1, further comprising a handle at said proximal end of said elongated body for actuating said delivery system.

23. The delivery system according to claim 22, wherein said actuating comprises one or more of:
a. actuating said at least two bending portions;
b. moving axially said elongated body;
c. moving axially said inner tube;
d. said actuating is performed manually by actuating one or more knobs;
e. said actuating is performed electronically by activating one or more motors.

24-28. (canceled)

29. The delivery system according to claim 22, wherein said handle comprises a quick release knob in communication with said elongated body and configured to allow rotation of said elongated body along its longitudinal axis by rotating of said quick release knob.

30. (canceled)

31. A method of delivering a prosthetic heart valve with a delivery system in a valve area located between an atrial space and a ventricular space,
said delivery system comprising: an elongated body having a distal end comprising an operational distal end including said prosthetic heart valve in a crimped configuration,
a virtual chord being defined between two locations adjacent to said operational distal end; said virtual chord having a length;
the method comprising:
a. inserting said distal end into a heart of a subject;
b. positioning said operational distal end in said ventricular space passing through said atrial space and said valve area;
c. retracting said operational distal end into said valve area by deforming at least one portion of said elongated body adjacent to said operational distal end; said deforming shortens said length of said virtual chord; wherein said deforming aligns said operational distal end ±20 degrees perpendicular to an opening of said heart valve.

32. The method according to claim 31, wherein said at least one portion of said elongated body is located in said atrium space while said deforming.

33. The method according to claim 31, wherein said positioning said operational distal end in said ventricular space comprises completely positioning said operational distal end in said ventricular space.

34. The method according to claim 31, wherein said method further comprises actuating said operational distal end for releasing said prosthetic heart valve in said valve area.

35. The method according to claim 34, wherein said actuating comprises one or more of retracting proximally said elongated body and pushing distally a prosthetic heart valve holder located within said operational distal end.

36. (canceled)

37. The method according to claim 34, wherein said releasing comprises one or more of:
a. releasing a plurality of anchors of said prosthetic heart valve into said valve space at said ventricular space, while holding the rest of said prosthetic heart valve in said operational distal end;
b. after said releasing of said plurality of anchors, releasing one or more prosthetic heart valve leg holders, while holding at least one of said prosthetic heart valve leg holders of said prosthetic heart valve in said operational distal end;
c. manipulating said elongated body for further positioning said prosthetic heart valve in said valve area;
d. releasing said at least one of said prosthetic heart valve leg holders of said prosthetic heart valve, thereby completely releasing said prosthetic heart valve from said operational distal end and in said valve area.

38-40. (canceled)

41. The method according to claim 31, wherein said deforming comprises one or more of bending one or more location in said elongated body and bending two distinct locations in said elongated body; and wherein said bending two distinct locations in said elongated body comprises bending two distinct location having opposite bending direction from each other.

42-50. (canceled)
